(19) United States
(12) Patent Application Publication
(10) Pub. No.: US 2022/0015339 A1
(43) Pub. Date: Jan. 20, 2022

(54) SYSTEMS AND BREEDING METHODS FOR PEST CONTROL

(71) Applicant: REGENTS OF THE UNIVERSITY OF MINNESOTA, Minneapolis, MN (US)

(72) Inventors: Maciej Bozyslaw Maselko, Sydney (AU); Przemyslaw Bajer, New Brighton, MN (US); Siba Ranjan Das, Falcon Heights, MN (US); James Parker, Minneapolis, MN (US); Michael Joseph Smanski, Falcon Heights, MN (US); Ambuj Upadhyay, Falcon Heights, MN (US)

(21) Appl. No.: 17/288,628

(22) PCT Filed: Nov. 5, 2019
(86) PCT No.: PCT/US2019/059826

§ 371 Date: Apr. 26, 2021

Related U.S. Application Data
(60) Provisional application No. 62/928,612, filed on Oct. 31, 2019.
(60) Provisional application No. 62/909,536, filed on Oct. 2, 2019.
(60) Provisional application No. 62/755,939, filed on Nov. 5, 2018.

(51) Int. Cl. A01K 67/027; A01K 67/033

(52) U.S. Cl. CPC A01K 67/0275; A01K 67/0333; A01K 2227/706; A01K 2227/40; A01K 2217/072

(57) ABSTRACT

A system for controlling population of a biological species includes a population of genetically-modified individuals of the biological species, where both males and females in the genetically-modified population carry two mutations. The first mutation is a repressible genetic mutation that results in death of a juvenile individual of the first sex when the juvenile individual of the first sex comprises the repressible lethal mutation and is reared in the absence of a repressor or causes an individual to be sterile when reared in the absence of a repressor. The second mutation is an underdominant genetic mutation.

### CROSS-REFERENCE TO RELATED APPLICATION

This application claims the benefit of U.S. Provisional Patent Application No. 62/755,939, filed Nov. 5, 2018, which is incorporated herein by reference in its entirety.

### GOVERNMENT FUNDING

This invention was made with government support under Grant No. HR0011836772 awarded by the Department of Defense/Defense Advanced Research Projects Agency (DARPA). The government has certain rights in the invention.

### SUMMARY

This disclosure describes, in one aspect, a system for controlling population of a biological species. Generally, the system includes a population of genetically-modified individuals of the biological species, where both males and females in the genetically-modified population carry two mutations. The first mutation is a repressible genetic mutation that results in death of a juvenile individual of the first sex when the juvenile individual of the first sex comprises the repressible lethal mutation and is reared in the absence of a repressor or causes an individual to be sterile when reared in the absence of a repressor. The second mutation is an underdominant genetic mutation. These two mutations work in concert so that offspring of a mating between a member of the system and a wild-type member of the biological species are nonviable and offspring of a mating between two members of the system results in viable offspring only of the second sex.

In some embodiments, the repressible mutation can result in female lethality or female sterility.

In some embodiments, the repressible mutation can result in male lethality or male sterility.

In some embodiments, the underdominant mutation can be a synthetic incompatibility mutation.

In another aspect, this disclosure describes a method for controlling the population of a wild biological species. Generally, the method includes releasing into a wild population of the wild biological species a system for controlling the population of the wild biological species, in which the system includes a population of genetically-modified individuals of the biological species. The system can include any embodiment of the genetically-modified population summarized above.

In some embodiments, the combination of the repressible mutation and the underdominant mutation reduces the population the wild biological species synergistically compared to using either the repressible mutation alone or the underdominant mutation alone.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Example of using a self-stocking incompatible male system (SSIMS) for limiting population expansion. (A) Wild type fish (left) cannot produce viable offspring when mated to SSIMS fish (right). SSIMS fish can only produce males when mated to each other in a wild environment. (B) Release of male and female SSIMS fish results in the expansion of SSIMS population via the production of males. Upon death of the stocked female(s), the SSIMS population is left with only males that cannot reproduce.

FIG. 2. Example of releasing SSIMS males for biocontrol. (A) Both male and female SSIMS mosquitoes can be reared in the laboratory in the presence of a repressor of the female lethal components (e.g., tetracycline). Only males reach maturity in the absence of the repressor. (B) SSIMS mosquito eggs or larva can be released into a wild environment. SSIMS males will reach maturity and suppress wild-type populations via the combined mechanisms of synthetic incompatibility and female lethality.

FIG. 3. Agent based model showing different interventions to control the invasive common carp. Simulations were performed in a hypothetical 100 ha lake for a one-time release of genetically modified fish. Initial fish population was reduced to 1 carp/ha using existing control methods, followed by no management or restocking with 20 carp/ha of various genetic background aimed at controlling the carp population. (A) Under ideal conditions, GD and SSIMS outperform other genetic interventions to control population growth of common carps. (B) When incorporating a promoter mutation rate of 0.001 for SSIMS and NHEJ rate of 0.001 for GD, SSIMS out-performs GD.

FIG. 4. Agent based modeling of Aedes aegypti populations. (A) Starting population of 100 wild-type mosquitoes, no control strategy. (B) Starting population of 100 wild-type mosquitoes and 500 (left) or 1000 (right) SI male mosquitoes. (C) Starting population of 100 wild-type mosquitoes and 500 (left) or 1000 (right) FL male mosquitoes. (D) Starting population of 100 wild-type mosquitoes and 500 (left) or 1000 (right) SSIMS mosquitoes of both sexes. In every simulation, starting populations are spread over every life-stage with 80% in egg, larval, or pupal stages and 20% in adult stages.

FIG. 5. Design and chromosomal location of female lethal circuit. Left panel, genetic design of FL constructs in two copy DmXFL12^{tTA} flies. FL1 and FL2 have only one copy of the X-linked FL construct on their X-chromosome. Right panel, chromosomal location of FL1 and FL2 insertion sites.

FIG. 6. Genotype and chromosomal location of EGI circuit. Left panel, proximity of sgRNA binding sites to transcription start site (TSS) for DmEGI^{pyr} strain. Sequences of both sgRNA binding sites (SEQ ID NO:4, SEQ ID NO:5, SEQ ID NO:6, and SEQ ID NO:7) are shown below promoter illustration, with protospacers in red and protospacer adjacent motifs in blue. Sequences of the mutated promoters at the sgRNA binding loci are shown below with differences highlighted in grey shadow. Right panel, chromosomal locations of genome alterations.

FIG. 7. Characterization of SSIMS flies. Image of flies showing presence of the two components, female lethal and EGI as required for functioning of SSIMS. Image on top is taken under bright field and that of bottom is taken under fluorescent light. Female lethal construct is characterized by the presence of GFP (left fly) and EGI is marked with red color in the eyes (middle fly). Note the presence of both red eye and GFP in the right fly indicating presence of both female lethal and EGI components (right fly).

FIG. 8. Characterization of SSIMS by mating among themselves and with wildtype (WT) flies in the presence or absence of the repressor (tetracycline).

### DETAILED DESCRIPTION OF ILLUSTRATIVE EMBODIMENTS

This disclosure describes a method of combining an underdominance system (e.g., engineered genetic incompatibility, EGI also known as synthetic incompatibility, SI) with a system that precludes the reproduction of a single sex) in sexually reproducing organisms (e.g., female lethality, FL, or a similarly regulated ‘daughterless’ system). The combined platform allows for various strategies for population control of undesirable species. The method described herein generally involve combining synthetic incompatibility with female lethal techniques to produce a platform with improved scalability and evolutionary robustness compared to previous platforms.

Populations of sexually reproducing pest organism such as disease vectoring mosquitoes or ecologically damaging invasive carp can cause substantial human economic losses and or health risks. Many strategies have been used to help reduce the burden of these organisms (e.g., insecticides, netting, sterile insect technique, synthetic incompatibility, female-lethality, gene-drives, etc.). The success of these strategies has been limited by, for example, challenges in scaling, high rates of escape mutations, and/or producing unintended harmful consequences for non-targeted beneficial organisms.

Genetic biocontrol strategies can produce highly specific and effective forms of population control. These strategies rely on the release of genetically engineered versions of the target species, which then mate with their wild counterparts. The product of this mating may result in no offspring, offspring of a single sex, offspring that are sterile, or offspring that carry a selfish genetic element (e.g., gene-drives, Madea elements) that spreads throughout the population and ultimately results in the population's extirpation/extinction.

Despite their potential, current genetic biocontrol strategies suffer multiple weaknesses. Methods that involve only the release of engineered male organisms that cannot produce viable offspring with wild females such as EGI, or Release of Insect carrying Dominant Lethality (RIDL), or some other underdominance approach require the efficient sorting of males from females. This sorting usually requires rearing both males and females to a life stage where they can be distinguished. This wastes resources on organisms that are never released and severely limits the scale at which production is feasible. Female lethal (FL) technologies allow for the control of female viability at young live stages, usually through the repression of a toxic gene by a small molecule (e.g., tetracycline). In some instances, adult females no longer require the small molecule for survival.

FL can be used as a tool to sort males carrying an FL genetic construct for release into the wild. Male carriers of FL will not produce female offspring in the wild. However, the larva of male offspring can still cause damage in the case of many agricultural pests such as, for example, the Diamondback moth. Furthermore, the male offspring can produce some females in subsequent generations as the FL constructs are diluted with each generation. EGI, RIDL, FL, or any other similar method also suffer from the need to release very large numbers of organisms to reduce wild pest population numbers.

Gene-drives (GD) are selfish genetic elements that are inherited in a non-Mendelian manner. In an organism that is hemizygous for a GD, the GD will be copied into the homologous chromosome that lacks the GD. Homozygosity for the GD elements may be lethal when disrupting an essential gene or cause sterility when disrupting a gene necessary for fertility. However, this process may happen in only a subset of cells, such as germ cells, so that the somatic tissue of an organism is hemizygous, yet all of the haploid gametes carry the GD. The advantage of GDs is that a small number of released organisms have the potential to affect large wild populations. A disadvantage of GDs, if they work as intended, is that it may be difficult to curtail their spread beyond a limited area or to stop them if there are unexpected and undesirable consequences of eliminating a pest organism. Current GDs in consideration of population control use programmable nucleases and homology-directed DNA repair as part of their copy mechanism. These suffer from very high rates of failure due to DNA repair via the non-homologous end-joining pathway which introduces mutations that prevent the GDs ability to spread.

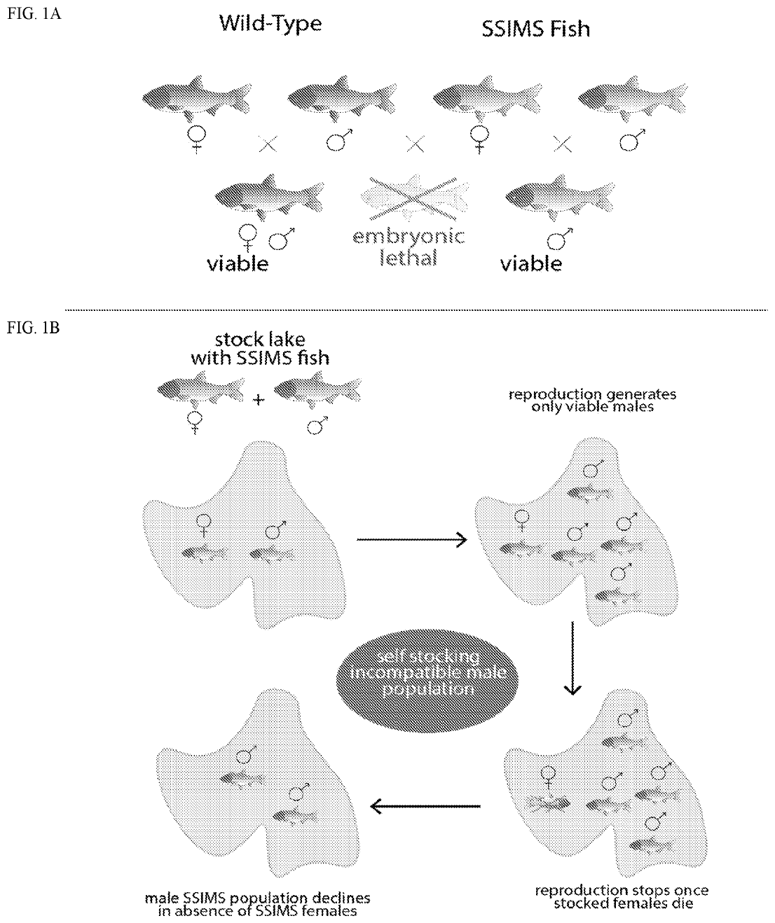

In contrast to the conventional genetic biocontrol strategies summarized above, this disclosure describes a method of combining an underdominance system (e.g., synthetic incompatibility) with a system that precludes the reproduction of a single sex (e.g., female lethality) in sexually reproducing organisms. The combined system results in a self-stocking incompatible male system (SSIMS) (FIG. 1).

While illustrated in FIG. 1 and described in detail below in the context of an exemplary embodiment in which the underdominance system is engineered genetic incompatibility and the system that precludes the reproduction of a single sex is female lethality, the genetic biocontrol methods described herein can be practiced using any suitable underdominance system combined with any suitable system that precludes the reproduction of a single sex. Suitable alternative underdominance systems include, for example, using siRNA, microRNA, dCas9, or dCas9-KRAB to reduce the expression of a single copy of a haploinsufficient gene. In a system for controlling the population of an insect, infection with select Wolbachia species for cytoplasmic incompatibility also may be suitable. Likewise, suitable systems that precludes the reproduction of a single sex include, for example, male lethality, female sterility, or male sterility systems.

In some instances, both male and female SSIMS adults can be released into a wild environment (FIG. 1B). A mating event between SSIMS and wild-type organisms will result in no viable offspring (FIG. 1A). A mating event between two SSIMS organisms, however, will produce only viable SSIMS males (FIG. 1A) in the absence of a repressor of the FL component.

SSIMS males may be generated in the wild for the duration of the reproductive life of SSIMS females. This produces limited amplification of engineered males (FIG. 1B) that can further suppress the wild pest population or perform some other function in the wild (e.g., bioremediation and/or biosensing).

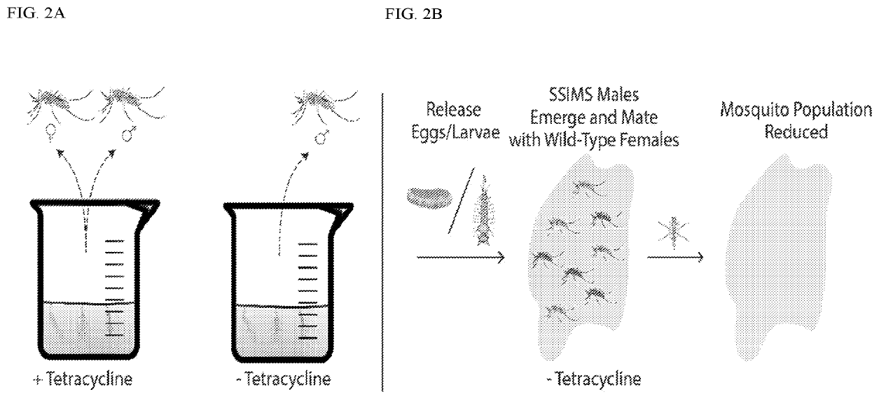

In other applications, SSIMS is used as a method to easily isolate SSIMS males that are genetically incompatible with wild females (FIG. 2). A reproductive population of SSIMS organisms can be maintained in a laboratory/production facility by supplying the necessary signal to repress the FL components of the system (FIG. 2A). The generation of SSIMS males for release is accomplished by transferring eggs/larva/juvenile SSIMS organisms to further develop in the absence of a FL repressor (FIG. 2A). The result is that only males will reach maturity while the females will die. The SSIMS males can then be released into the wild (FIG. 2B) where there is an absence of the FL repressor. Again, mating event between SSIMS male and a wild-type female produces no viable offspring (FIG. 1A). In FIG. 2B, the wild lacks the FL repressor, so no SSIMS female offspring are produced, so there is no opportunity for a sustained SSIMS population

SSIMS can be applied to any organisms that have male and female sexes. Exemplary animals into which SSIMS may be introduced can include, for example, an insect (e.g., mosquito, tstetse fly, spotted-wing drosophila, diamondback moth, new world screwworm, Mediterranean fruit-fly, olive fly, gypsy moth, codling moth, deer tick, etc.), a fish (e.g., salmon, carp, sea lamprey, etc.), a mammal (e.g., swine, a mouse, a rat, etc.), an amphibian (e.g., a cane toad, a bullfrog, etc.), a reptile (e.g., brown tree snake, etc.), or a crustacean (e.g., rusty crayfish, etc.).

The current examples involve males reaching maturity in the absence of a repressor of a toxic gene. In some embodiments, however, it may be desirable to only release females. In such cases, a male lethality mechanism can be used.

Simulations of population control in common carp (Cyprinus carpio) were performed using an agent-based model specific to common carp biology (Bajer et al., 2015. Oikos 124(11): 1520-1526). These simulations are simply examples that demonstrate the performance of different genetic interventions and should not be interpreted as the only possible scenarios.

All simulations involved the release of sexually mature, three-year-old fish. For the GD simulation, a fish that is heterozygous for the GD will have 100% GD gametes and a fish homozygous for GD will be non-viable. The FL simulation involves the release of males and female fish. Any female born in the wild that carriers the FL system will be non-viable. The EGI and SSIMS fish produce non-viable offspring when crossed with wild-type fish. The SSIMS fish only produce males when crossed with SSIMS fish in the wild; all females die at larval stage. Simulations are performed on a hypothetical 100 ha lake. Simulated mutations in EGI or SSIMS scenarios generated wild-type fish that are capable of hybridizing with the engineered fish due to a mutation in the targeted promoter. Simulated mutations in the GD scenarios resulted in alleles that could not be targeted by the GD nuclease for copying by homology-directed repair. Of these, two-thirds where lethal if homozygous or combined with a GD allele; the remaining one-third of the mutations had no impact on the fish beyond resistance to the GD nuclease. All carp simulations assumed that standard seining practice was performed in addition to a genetic control strategy. Seining reduces the number of engineered fish needed for control, however, it may not be necessary in all cases.

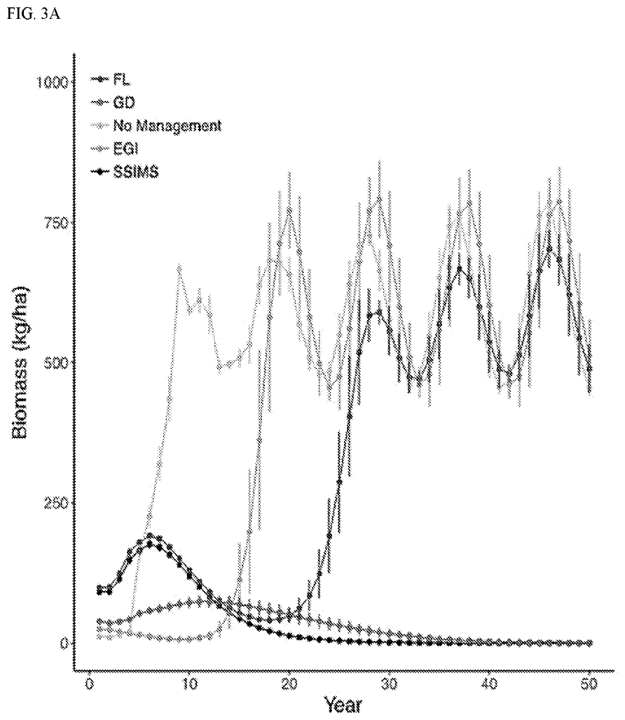

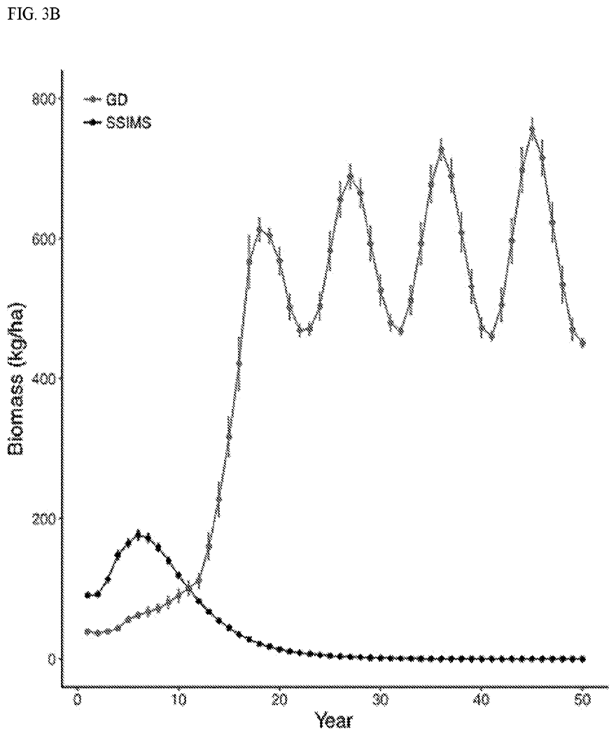

Simulations started following reduction in concentration of fish in the lake to about 1 carp/ha, which can be achieved by conventional management practices such as, for example, netting or the use of a piscicide. An optimum restocking number and male-female ratio was earlier identified by running several simulations with different release numbers and male-female ratio. Data shown in FIG. 3 is simulated with onetime release of 2040 fish containing 100% males for EGI or 60% males for other genetically modified fish. Results are average of 10 simulations for each of the different conditions.

Results show that in the presence of no mutations, a one-time release of FL, or EGI fish perform marginally better than no genetic management strategy. Genetic conditions that effectively control fish population to extinction are GD or SSIMS. Since GD intervention are susceptible escape mutations from non-homologous end joining (NHEJ), and SSIMS are, in theory, susceptible to escape mutations in the targeted promoter region, these two conditions were simulated with a relatively conservative rate of 1 in a 1000. The simulation showed that SSIMS intervention is more resistant to promoter mutation while GD interventions are very sensitive to NHEJ. SSIMS is therefore a both an effective and robust method for control of wild populations.

An agent-based model was created to simulate a population of Aedes aegypti mosquitoes, based on a previously described model that incorporates density-dependent mortality for eggs and larvae in the first two instars (Dye, C., 1984. Journal of Animal Ecology 53(1):247-268). Values for average egg production per female, gonotrophic cycle length, and adult mortality per time step were obtained from published experimental results (Goindin et al., 2015. PLoS ONE, 10(8)).

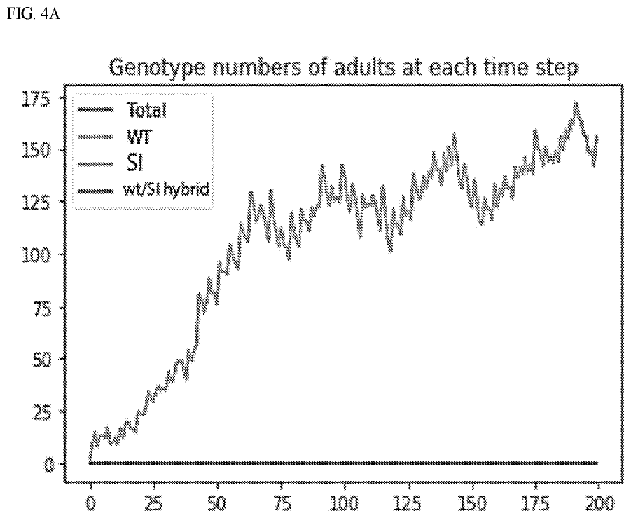

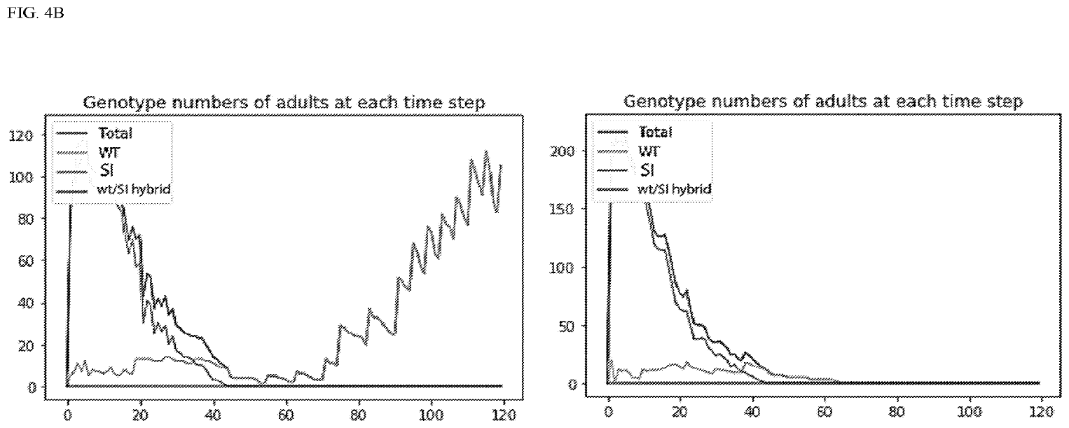

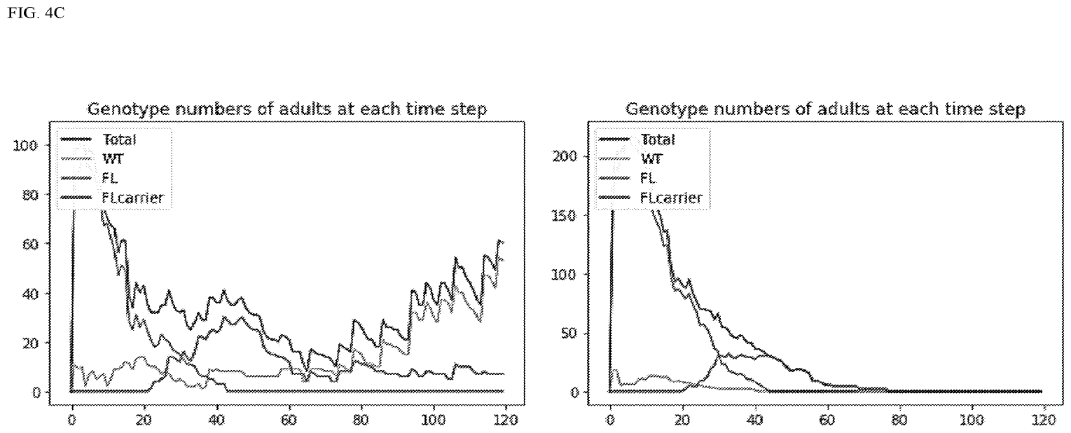

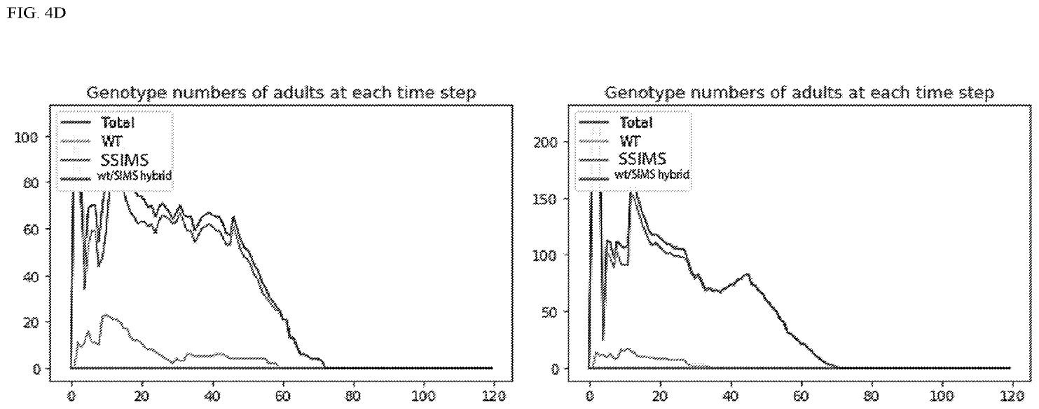

The simulations described in FIG. 4 represent a real-life scenario in which wild populations are suppressed with a conventional control strategy (or early season scenarios in locations where mosquitoes are seasonal) and controlled with release of GMO animals. All GMO strategies were tested at 5× and 10× release numbers compared to wild-type. With no control strategy, the numbers of wild-type adults quickly increase to a steady state of between 100 and 150 individuals after 50 time-steps (roughly 75 days).

Each of the genetic control strategies were effective when control animals were released at 10× population numbers. Notably, the SSIMS strategy eliminated wild-type adults completely after only 35 time steps, compared to 40 time steps for female lethal and 60 time steps for engineered genetic incompatibility alone. When GMO animals were released at 5× wild-type population numbers, only the SSIMS approach effectively eradicated wild-type animals. The wild-type adult population numbers went to zero after 60 time steps, while wild adult populations grew at a rate that mirrors the no treatment control after an initial suppression period for either single genetic control strategy. Based on the numbers of GMO adults produced in each experiment, it appears that the reason the combined approach outperformed the others is that incompatible adults were present for twice as long. By releasing both sexes of the SSIMS mosquitoes, two generations of incompatible mosquitoes were produced. This simulation allowed for the release of mosquitoes across multiple life stages. Initial SSIMS females survive larval stages via addition of repressor of FL.

Thus, this disclosure describes a system for controlling population of a biological species. The system uses genetically-modified individuals of the biological species designed so that the offspring of matings between the genetically-modified individuals and breed wild individuals of the biological are nonviable.

Generally, the genetic modifications involve two separate genetic modifications that, in concert with one another, can provide synergistic control of the wild population of the biological species compared to systems that use either of the genetic modifications alone. The first genetic modification involves using a repressible genetic mutation that produces lethality or sterility in one sex when an individual of that sex harbors the genetic mutation and is reared in an environment that lacks the repressor. The repressible genetic mutation has no effect on members of the other sex even when reared in the absence of the repressor. In practice, this allows one to rear individuals of both sexes under a controlled environment that includes the repressor—e.g., tetracycline—and then release the mature individuals into a wild environment that lacks the repressor. Since absence of the repressor affects only juveniles, the released individuals are viable and are able to mate with wild individuals (or with other genetically-modified individuals). As used herein, the term “juvenile” refers to an individual who has not yet reached an age of sexual maturity. Offspring of matings that occur in the wild—e.g., in the absence of the repressor—and that harbor the repressible genetic mutation will be affected and will either be sterile or will die, depending on whether the repressible genetic mutation induces sterility or lethality.

The second genetic mutation is an underdominance mutation in which the heterozygous state induces a selective disadvantage (e.g., death, sterility, etc.). Thus, matings in the wild between the genetically-modified individuals of the system population and wild individuals will produce nonviable offspring. Homozygous individuals harboring the underdominance genetic mutation are viable, but since there is no repressor to repress the first mutation, matings in the wild between two individuals of the system population will produce viable offspring of only one sex. This feature of the system can produce self-regulation of the system as the supply of individuals harboring both mutation will eventually die off.

The genetic mutations can be incorporated into any suitable species that reproduces sexually and whose population one wishes to control. The genetic mutations may be introduced into individuals of the biological species using conventional method to introduce each genetic mutation into individuals of the biological species to produce the system population. Generally, the biological species can be a pest species or an invasive species. In certain embodiments, the biological species can be a fish or an insect.

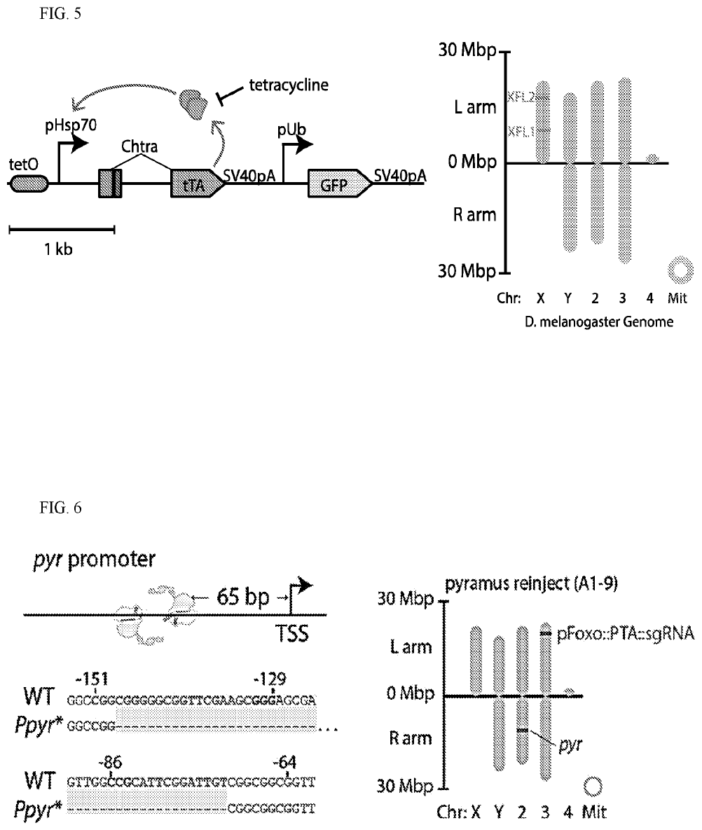

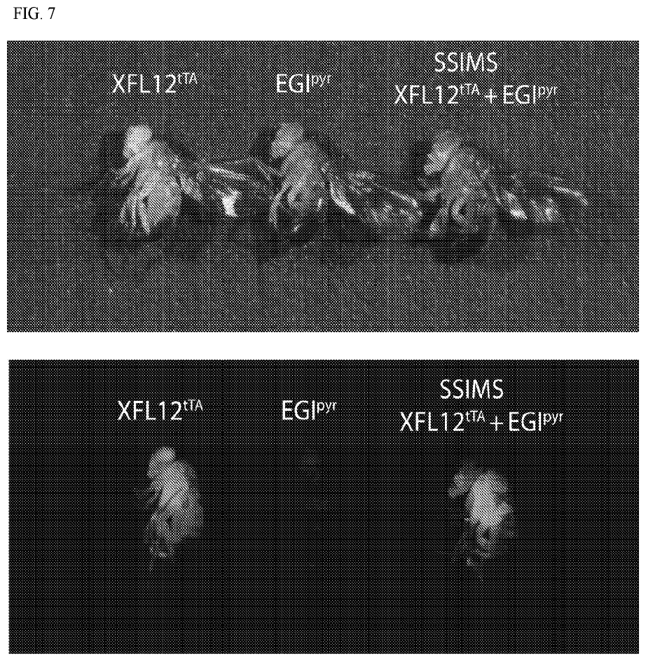

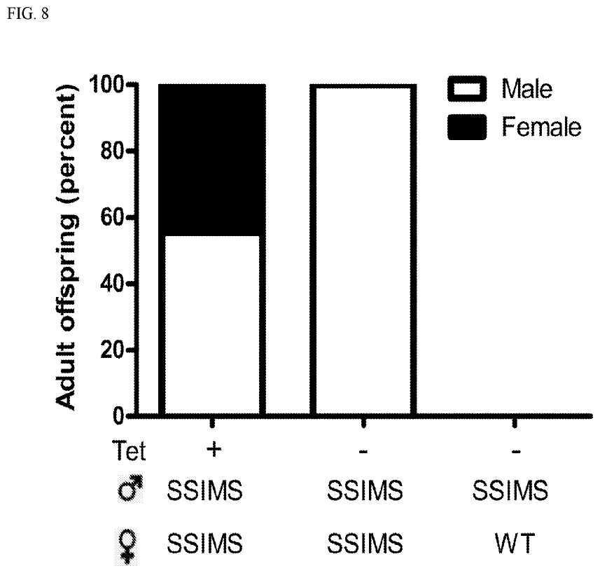

In one exemplary embodiment, the SSIMS organisms can be created using the model eukaryote, D. melanogaster. The exemplary SSIMS flies were created by combining two distinct genetic technologies together: engineered genetic incompatibility (e.g., synthetic incompatibility) and sex sorting (e.g., female lethality). In the exemplary embodiment, flies were created containing a female lethality system (FIG. 5) so that only male progeny are produced. In the exemplary embodiment, the flies also contained engineered genetic incompatibility (FIG. 6) to eliminate the production of viable progeny when crossed with wild-type individuals. The SSIMS flies produced contained both genetic systems, indicating that both genetic constructs were compatible with each other without any appreciable fitness cost (FIG. 7). The SSIMS approach can be used for biocontrol purpose since it allows creation of male only progeny outside of the lab environment, in absence of a repressor (e.g., tetracycline) (FIG. 8). Mating between SSIMS and wild-type flies leads to no offspring, which will break the reproduction cycle of wild females leading to control of population.

While exemplified in the context of an embodiment in which sex sorting is accomplished using a female lethality construct, SSIMS organisms and methods described herein can involve any known, conventional sex sorting genetic strategy. Exemplary sex sorting genetic technologies are described in, for example, U.S. Pat. No. 10,376,925; International Publication No. WO 2006/060603 A1; and U.S. Provisional Patent Application No. 62/909,536, (filed Oct. 2, 2019, entitled “SYSTEMS AND METHODS FOR BATCH CULTIVATION OF NON-TRANSGENIC HETEROGAMETES”).

While exemplified in the context of an embodiment in which the SSIMS fail to produce viable progeny due to engineered genetic incompatibility, SSIMS organisms and methods described herein can involve other synthetic incompatibility strategies that make SSIMS organisms incapable of producing viable progeny when crossed with wild-type organisms. Exemplary alternative strategies for producing synthetic incompatibility include, for example, underdominant systems of insect control (Reed, F. A., 2014, PLoS One 9:e97557), precision guided sterile insect technique (Kandul et. al., 2019, Nat Comm 10:84, doi:10.1038/s41467-018-07964-7), and those described in U.S. Patent Publication No. 2018/0327762 A1, International Publication No. WO 2018/209014 A1, U.S. Provisional Patent Application No. 62/928,612 (filed Oct. 31, 2019, entitled “SYSTEMS AND METHODS FOR GENERATING GENETIC INCOMPATIBILITY”),

While exemplified in the context of D. melanogaster, SSIMS organisms may be constructed from any suitable multicellular organism. Exemplary plants into which the SSIMS system may be introduced can include, for example, a field crop (e.g., tobacco, corn, soybean, rice, etc.), a tree (e.g., poplar, rubber tree, etc.), or turfgrass (e.g. creeping bentgrass). Exemplary animals into which the SSIMS system may be introduced can include, for example, an insect (e.g., mosquito, tsetse fly, spotted-wing drosophila, olive fly, gypsy moth, codling moth, deer tick, etc.), a fish (e.g., salmon, carp, sea lamprey, etc.), a mammal (e.g., swine, a mouse, a rat, etc.), an amphibian (e.g., a cane toad, a bullfrog, etc.), a reptile (e.g., brown tree snake, etc.), or a crustacean (e.g., rusty crayfish, etc.).

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Plasmids

Plasmid expressing dCas9-VPR was constructed by Gibson assembly combining NotI linearized pMBO2744 attP vector backbone with dCas9-VPR PCR amplified from pAct:dCas9-VPR (Addgene #78898) and SV40 terminator for pH-Stinger (BDSC) to generate pMM7-6-1 (SEQ ID NO:1). Gibson assembly was used to clone 5′UTR and approximately 1.5 kb of promoter sequence pFoxO into Nod linearized pMM7-6-1 to create pMM7-6-2 (SEQ ID NO:2).

Plasmid expressing both sgRNAs and dCas9-VPR were generated by assembling amplified sgRNA cassettes targeting pyr (BDSC stock #67537) into KpnI linerarized plasmid pMM7-6-2 to create plasmid pAH1 (SEQ ID NO:3).

The tetracycline female lethal circuit was made by adapting a previously described female-lethal piggybac vector, pB[FL3] (Li et al., 2014, Insect Biochem. Mol. Biol. 51:80-88). The final plasmid, pMM7-8-1 (SEQ ID NO:8), was made by transferring the lethal circuit to pUB-EGFP (Schetelig et al., 2009, Proc. Natl. Acad. Sci. U.S.A. 106:18171-18176), which contains an attB site for ΦC31-mediated integration and ubiquitin promoter driven EGFP expression.

### Generating and Maintaining Transgenic Drosophila Strains

D. melanogaster strains were maintained at 25° C. and 12-hour days in cornmeal agar (NUTRI-FLY, Genesee Scientific Corp., El Cajon, Calif.) supplemented with 10-200 μg/ml tetracycline, as necessary. Experimental crosses were performed at 25° C. and 12-hour days. Existing Cas9 and sgRNA strains were obtained from the Bloomington Drosophila Stock Center (Bloomington, Ind.). All transgenic flies were generated via ΦC31-mediated integration targeted to attP landing sites. Embryo microinjections were performed by BestGene Inc. (Chino Hills, Ca).

Transgenic EGI D. melanogaster strain, DmEGI^{pyr} was generated by microinjection and ΦC31 mediated integration of pAH-1 into the 2^{nd} chromosome attP site of y[1] w[1118]; PBac{y[+]-attP-3B}VK00037; +(BDSC #9752). Transgenic animals were isolated by crossing to balancers present on 2^{nd} (CyO-GFP) and 3^{rd} (Tm2 and Tm6) chromosome, then homozygosed by selecting non-balancer animals to generate true breeding strain.

Transgenic female lethal strains were generated by microinjection and ΦC31 integration of pMM7-8-1 into the X-chromosome at attP sites y[1] w[*] P {y[+t7.7]=CaryIP}su(Hw)attP8 (BDSC #32233) to create DmXFL1^{tTA} or y[1]w[1118]pBac{y[+]-attP-9A}VK00006 (BDSC #9726) to create DmXFL2^{tTA}. Transgenic animals were isolated by crossing to FM6 balancers, then homozygosed by selecting non-balancer animals to generate true breeding strain. DmXFL12^{tTA} flies were created by isolating recombinant chromosome of DmXFL1^{tTA} and DmXFL2^{tTA} and screening for the presence of transgene in both locations of the X-chromosome.

SSIMS flies were generated by mating female lethal flies, DmXFL12^{tFA} and DmEGI^{pyr} together by using balancer chromosome strains that prevent recombination across chromosomes. The following balancer chromosomes were used: FM7-GFP on X, CyO-GFP on 2, Tm2 and Tm6 on 3.

### Mating Compatibility Tests

Genetic compatibility was assayed between parental stock homozygous for SSIMS and between SSIMS and wild-type flies. Test crosses were performed by crossing sexually-mature adult males to sexually-mature respective virgin females. The adults were removed from the vials after five days and the offspring were counted after fifteen days.

### Example 2

SSIMS Cyprinus carpio are produced by engineering Female Lethal (FL) components into the fish as previously described (Thomas et al., 2000. Science 287:2474-2476; Concha et al., 2016. BMC Biol. 14:72; Thresher et al., 2014. Nat. Biotechnol. 32:424-427; Fu et al., 2010. Proc. Natl. Acad. Sci. 107:4550-1554).

The transformed fish are further modified by introducing a synthetic incompatibility (SI) components as previously described (Reed, F. A., 2014. PLoS One 9:e97557; Maselko et al., 2017. Nat. Commun. 8:883; Aliota et al., 2016. Sci. Rep. 6:28792).

### Example 3

SSIMS Aedes aegypti are produced by engineering Female Lethal (FL) components into the mosquitoes as previously described (Thomas et al., 2000. Science 287:2474-2476; Concha et al., 2016. BMC Biol. 14:72; Thresher et al., 2014. Nat. Biotechnol. 32:424-427; Fu et al., 2010. Proc. Natl. Acad. Sci. 107:4550-4554).

The transformed mosquitoes are further modified by introducing a synthetic incompatibility (SI) components as previously described (Reed, F. A., 2014. PLoS One 9:e97557; Maselko et al., 2017. Nat. Commun. 8:883; Aliota et al., 2016. Sci. Rep. 6:28792).

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

SEQUENCE LISTING FREE TEXT

SEQ ID NO: 1-pMM7-6-1
CCACNCACGTTTCGTAGTTGCTCTTTCGCTGTCTCCCACCCGCTNTCCGCAACACATTCACCTTTTGTTC
GACGACCNTNGGAGCGACTGTCGTTAGTTCCGCGCGATTCGGTTCGCTCAAATGGTTCCGAGTGGTTCAT
TTCGTCTCAATAGAAATTAGTAATAAATATTTGTATGTACAATTTATTTGCTCCAATATATTTGTATATA
TTTCCCTCACAGCTATATTTATTCTAATTTAATATTATGACTTTTTAAGGTAATTTTTTGTGACCTGTTC
GGAGTGATTAGCGTTACAATTTGAACTGAAAGTGACATCCAGTGTTTGTTCCTTGTGTAGATGCATCTCA
AAAAAATGGTGGGCATAATAGTGTTGTTTATATATATCAAAAATAACAACTATAATAATAAGAATACATT
TAATTTAGAAAATGCTTGGATTTCACTGGAACTAGGCTAGCATAACTTCGTATAATGTATGCTATACGAA
GTTATGCTAGCGGATCCGGGAATTGGGAATTCACGTAAGTACTGTCTGCAGCGTAAGCTTCGTACGTAGC
GGCCGCAATCTTACAAAATGGACAAGAAGTACTCCATTGGGCTCGCTATCGGCACAAACAGCGTCGGCTG
GGCCGTCATTACGGACGAGTACAAGGTGCCGAGCAAAAAATTCAAAGTTCTGGGCAATACCGATCGCCAC
AGCATAAAGAAGAACCTCATTGGCGCCCTCCTGTTCGACTCCGGGGAGACGGCCGAAGCCACGCGGCTCA
AAAGAACAGCACGGCGCAGATATACCCGCAGAAAGAATCGGATCTGCTACCTGCAGGAGATCTTTAGTAA
TGAGATGGCTAAGGTGGATGACTCTTTCTTCCATAGGCTGGAGGAGTCCTTTTTGGTGGAGGAGGATAAA
AAGCACGAGCGCCACCCAATCTTTGGCAATATCGTGGACGAGGTGGCGTACCATGAAAAGTACCCAACCA
TATATCATCTGAGGAAGAAGCTTGTAGACAGTACTGATAAGGCTGACTTGCGGTTGATCTATCTCGCGCT
GGCGCATATGATCAAATTTCGGGGACACTTCCTCATCGAGGGGGACCTGAACCCAGACAACAGCGATGTC
GATAAACTCTTTATCCAACTGGTTCAGACTTACAATCAGCTTTTCGAAGAGAACCCGATCAACGCATCCG
GAGTTGACGCCAAAGCAATCCTGAGCGCTAGGCTGTCCAAATCCCGGCGGCTCGAAAACCTCATCGCACA
GCTCCCTGGGGAGAAGAAGAACGGCCTGTTTGGTAATCTTATCGCCCTGTCACTCGGGCTGACCCCCAAC
TTTAAATCTAACTTCGACCTGGCCGAAGATGCCAAGCTTCAACTGAGCAAAGACACCTACGATGATGATC
TCGACAATCTGCTGGCCCAGATCGGCGACCAGTACGCAGACCTTTTTTTGGCGGCAAAGAACCTGTCAGA
CGCCATTCTGCTGAGTGATATTCTGCGAGTGAACACGGAGATCACCAAAGCTCCGCTGAGCGCTAGTATG
ATCAAGCGCTATGATGAGCACCACCAAGACTTGACTTTGCTGAAGGCCCTTGTCAGACAGCAACTGCCTG
AGAAGTACAAGGAAATTTTCTTCGATCAGTCTAAAAATGGCTACGCCGGATACATTGACGGCGGAGCAAG
CCAGGAGGAATTTTACAAATTTATTAAGCCCATCTTGGAAAAAATGGACGGCACCGAGGAGCTGCTGGTA
AAGCTTAACAGAGAAGATCTGTTGCGCAAACAGCGCACTTTCGACAATGGAAGCATCCCCCACCAGATTC
ACCTGGGCGAACTGCACGCTATCCTCAGGCGGCAAGAGGATTTCTACCCCTTTTTGAAAGATAACAGGGA
AAAGATTGAGAAAATCCTCACATTTCGGATACCCTACTATGTAGGCCCCCTCGCCCGGGGAAATTCCAGA
TTCGCGTGGATGACTCGCAAATCAGAAGAGACCATCACTCCCTGGAACTTCGAGGAAGTCGTGGATAAGG
GGGCCTCTGCCCAGTCCTTCATCGAAAGGATGACTAACTTTGATAAAAATCTGCCTAACGAAAAGGTGCT
TCCTAAACACTCTCTGCTGTACGAGTACTTCACAGTTTATAACGAGCTCACCAAGGTCAAATACGTCACA
GAAGGGATGAGAAAGCCAGCATTCCTGTCTGGAGAGCAGAAGAAAGCTATCGTGGACCTCCTCTTCAAGA
CGAACCGGAAAGTTACCGTGAAACAGCTCAAAGAAGACTATTTCAAAAAGATTGAATGTTTCGACTCTGT
TGAAATCAGCGGAGTGGAGGATCGCTTCAACGCATCCCTGGGAACGTATCACGATCTCCTGAAAATCATT
AAAGACAAGGACTTCCTGGACAATGAGGAGAACGAGGACATTCTTGAGGACATTGTCCTCACCCTTACGT
TGTTTGAAGATAGGGAGATGATTGAAGAACGCTTGAAAACTTACGCTCATCTCTTCGACGACAAAGTCAT
GAAACAGCTCAAGAGGCGCCGATATACAGGATGGGGGCGGCTGTCAAGAAAACTGATCAATGGGATCCGA
GACAAGCAGAGTGGAAAGACAATCCTGGATTTTCTTAAGTCCGATGGATTTGCCAACCGGAACTTCATGC
AGTTGATCCATGATGACTCTCTCACCTTTAAGGAGGACATCCAGAAAGCACAAGTTTCTGGCCAGGGGGA
CAGTCTTCACGAGCACATCGCTAATCTTGCAGGTAGCCCAGCTATCAAAAAGGGAATACTGCAGACCGTT
AAGGTCGTGGATGAACTCGTCAAAGTAATGGGAAGGCATAAGCCCGAGAATATCGTTATCGAGATGGCCC
GAGAGAACCAAACTACCCAGAAGGGACAGAAGAACAGTAGGGAAAGGATGAAGAGGATTGAAGAGGGTAT
AAAAGAACTGGGGTCCCAAATCCTTAAGGAACACCCAGTTGAAAACACCCAGCTTCAGAATGAGAAGCTC
TACCTGTACTACCTGCAGAACGGCAGGGACATGTACGTGGATCAGGAACTGGACATCAATCGGCTCTCCG
ACTACGACGTGGCTGCTATCGTGCCCCAGTCTTTTCTCAAAGATGATTCTATTGATAATAAAGTGTTGAC
AAGATCCGATAAAGCTAGAGGGAAGAGTGATAACGTCCCCTCAGAAGAAGTTGTCAAGAAAATGAAAAAT
TATTGGCGGCAGCTGCTGAACGCCAAACTGATCACACAACGGAAGTTCGATAATCTGACTAAGGCTGAAC
GAGGTGGCCTGTCTGAGTTGGATAAAGCCGGCTTCATCAAAAGGCAGCTTGTTGAGACACGCCAGATCAC
CAAGCACGTGGCCCAAATTCTCGATTCACGCATGAACACCAAGTACGATGAAAATGACAAACTGATTCGA
GAGGTGAAAGTTATTACTCTGAAGTCTAAGCTGGTCTCAGATTTCAGAAAGGACTTTCAGTTTTATAAGG
TGAGAGAGATCAACAATTACCACCATGCGCATGATGCCTACCTGAATGCAGTGGTAGGCACTGCACTTAT
CAAAAAATATCCCAAGCTTGAATCTGAATTTGTTTACGGAGACTATAAAGTGTACGATGTTAGGAAAATG
ATCGCAAAGTCTGAGCAGGAAATAGGCAAGGCCACCGCTAAGTACTTCTTTTACAGCAATATTATGAATT
TTTTCAAGACCGAGATTACACTGGCCAATGGAGAGATTCGGAAGCGACCACTTATCGAAACAAACGGAGA
AACAGGAGAAATCGTGTGGGACAAGGGTAGGGATTTCGCGACAGTCCGGAAGGTCCTGTCCATGCCGCAG
GTGAACATCGTTAAAAAGACCGAAGTACAGACCGGAGGCTTCTCCAAGGAAAGTATCCTCCCGAAAAGGA
ACAGCGACAAGCTGATCGCACGCAAAAAAGATTGGGACCCCAAGAAATACGGCGGATTCGATTCTCCTAC
AGTCGCTTACAGTGTACTGGTTGTGGCCAAAGTGGAGAAAGGGAAGTCTAAAAAACTCAAAAGCGTCAAG
GAACTGCTGGGCATCACAATCATGGAGCGATCAAGCTTCGAAAAAAACCCCATCGACTTTCTCGAGGCGA
AAGGATATAAAGAGGTCAAAAAAGACCTCATCATTAAGCTTCCCAAGTACTCTCTCTTTGAGCTTGAAAA
CGGCCGGAAACGAATGCTCGCTAGTGCGGGCGAGCTGCAGAAAGGTAACGAGCTGGCACTGCCCTCTAAA
TACGTTAATTTCTTGTATCTGGCCAGCCACTATGAAAAGCTCAAAGGGTCTCCCGAAGATAATGAGCAGA
AGCAGCTGTTCGTGGAACAACACAAACACTACCTTGATGAGATCATCGAGCAAATAAGCGAATTCTCCAA
AAGAGTGATCCTCGCCGACGCTAACCTCGATAAGGTGCTTTCTGCTTACAATAAGCACAGGGATAAGCCC
ATCAGGGAGCAGGCAGAAAACATTATCCACTTGTTTACTCTGACCAACTTGGGCGCGCCTGCAGCCTTCA
AGTACTTCGACACCACCATAGACAGAAAGCGGTACACCTCTACAAAGGAGGTCCTGGACGCCACACTGAT
TCATCAGTCAATTACGGGGCTCTATGAAACAAGAATCGACCTCTCTCAGCTCGGTGGAGACAGCAGGGCT
GACCCCAAGAAGAAGAGGAAGGTGGAGGCCAGCGGTTCCGGACGGGCTGACGCATTGGACGATTTTGATC
TGGATATGCTGGGAAGTGACGCCCTCGATGATTTTGACCTTGACATGCTTGGTTCGGATGCCCTTGATGA
CTTTGACCTCGACATGCTCGGCAGTGACGCCCTTGATGATTTCGACCTGGACATGCTGATTAACTCTAGA
AGTTCCGGATCTCCGAAAAAGAAACGCAAAGTTGGTAGCCAGTACCTGCCCGACACCGACGACCGGCACC
GGATCGAGGAAAAGCGGAAGCGGACCTACGAGACATTCAAGAGCATCATGAAGAAGTCCCCCTTCAGCGG
CCCCACCGACCCTAGACCTCCACCTAGAAGAATCGCCGTGCCCAGCAGATCCAGCGCCAGCGTGCCAAAA
CCTGCCCCCCAGCCTTACCCCTTCACCAGCAGCCTGAGCACCATCAACTACGACGAGTTCCCTACCATGG
TGTTCCCCAGCGGCCAGATCTCTCAGGCCTCTGCTCTGGCTCCAGCCCCTCCTCAGGTGCTGCCTCAGGC
TCCTGCTCCTGCACCAGCTCCAGCCATGGTGTCTGCACTGGCTCAGGCACCAGCACCCGTGCCTGTGCTG
GCTCCTGGACCTCCACAGGCTGTGGCTCCACCAGCCCCTAAACCTACACAGGCCGGCGAGGGCACACTGT
CTGAAGCTCTGCTGCAGCTGCAGTTCGACGACGAGGATCTGGGAGCCCTGCTGGGAAACAGCACCGATCC
TGCCGTGTTCACCGACCTGGCCAGCGTGGACAACAGCGAGTTCCAGCAGCTGCTGAACCAGGGCATCCCT
GTGGCCCCTCACACCACCGAGCCCATGCTGATGGAATACCCCGAGGCCATCACCCGGCTCGTGACAGGCG
CTCAGAGGCCTCCTGATCCAGCTCCTGCCCCTCTGGGAGCACCAGGCCTCCCTAATGGACTGCTGTCTGG
CGACGAGGACTTCAGCTCTATCGCCGATATGGATTTCTCAGCCTTGCTGGGCTCTGGCAGCGGCAGCCGG
GATTCCAGGGAAGGGATGTTTTTGCCGAAGCCTGAGGCCGGCTCCGCTATTAGTGACGTGTTTGAGGGCC
GCGAGGTGTGCCAGCCAAAACGAATCCGGCCATTTCATCCTCCAGGAAGTCCATGGGCCAACCGCCCACT
CCCCGCCAGCCTCGCACCAACACCAACCGGTCCAGTACATGAGCCAGTCGGGTCACTGACCCCGGCACCA
GTCCCTCAGCCACTGGATCCAGCGCCCGCAGTGACTCCCGAGGCCAGTCACCTGTTGGAGGATCCCGATG
AAGAGACGAGCCAGGCTGTCAAAGCCCTTCGGGAGATGGCCGATACTGTGATTCCCCAGAAGGAAGAGGC
TGCAATCTGTGGCCAAATGGACCTTTCCCATCCGCCCCCAAGGGGCCATCTGGATGAGCTGACAACCACA
CTTGAGTCCATGACCGAGGATCTGAACCTGGACTCACCCCTGACCCCGGAATTGAACGAGATTCTGGATA
CCTTCCTGAACGACGAGTGCCTCTTGCATGCCATGCATATCAGCACAGGACTGTCCATCTTCGACACATC
TCTGTTTTGACCGACTCTAGATCATAATCAGCCATACCACATTTGTAGAGGTTTTACTTGCTTTAAAAAA
CCTCCCACACCTCCCCCTGAACCTGAAACATAAAATGAATGCAATTGTTGTTGTTAACTTGTTTATTGCA
GCTTATAATGGTTACAAATAAAGCAATAGCATCACAAATTTCACAAATAAAGCATTTTTTTCACTGCATT
CTAGTTGTGGTTTGCCCAAACTCATCAATGTATCTTAGCGGCTCGAGGGTACCTCTAGAGATCCACTAGT
GTCGACGATGTAGGTCACGGTCTCGAAGCCGCGGTGCGGGTGCCAGGGCGTGCCCTTGGGCTCCCCGGGC
GCGTACTCCACCTCACCCATCTGGTCCATCATGATGAACGGGTCGAGGTGGCGGTAGTTGATCCCGGCGA
ACGCGCGGCGCACCGGGAAGCCCTCGCCCTCGAAACCGCTGGGCGCGGTGGTCACGGTGAGCACGGGACG
TGCGACGGCGTCGGCGGGTGCGGATACGCGGGGCAGCGTCAGCGGGTTCTCGACGGTCACGGCGGGCATG
TCGACACTA
SEQ ID NO: 2-pMM7-6-2
CCACNCACGTTTCGTAGTTGCTCTTTCGCTGTCTCCCACCCGCTNTCCGCAACACATTCACCTTTTGTTC
GACGACCNTNGGAGCGACTGTCGTTAGTTCCGCGCGATTCGGTTCGCTCAAATGGTTCCGAGTGGTTCAT
TTCGTCTCAATAGAAATTAGTAATAAATATTTGTATGTACAATTTATTTGCTCCAATATATTTGTATATA
TTTCCCTCACAGCTATATTTATTCTAATTTAATATTATGACTTTTTAAGGTAATTTTTTGTGACCTGTTC
GGAGTGATTAGCGTTACAATTTGAACTGAAAGTGACATCCAGTGTTTGTTCCTTGTGTAGATGCATCTCA
AAAAAATGGTGGGCATAATAGTGTTGTTTATATATATCAAAAATAACAACTATAATAATAAGAATACATT
TAATTTAGAAAATGCTTGGATTTCACTGGAACTAGGCTAGCATAACTTCGTATAATGTATGCTATACGAA
GTTATGCTAGCGGATCCGGGAATTGGGAATTCACGTAAGTACTGTCTGCAGCGTAAGCTTCGTACGTAGC
GTCAAATTTGGTTGTGATTACGAGACGGAGACCGAGACGGCGACGACAGTTAGCCATTCGCCACGCGCCA
ACGCAAATGAAACGCTCTATACATATTTTTGTATATTTTCTGTTTTTTTTTGCCGCTGACAATTATGATC
AAGTATTAGCTGGCGATAGCTGAAACGTCTGTGTAATTTCAATGGAATGGAATGGGAAGTGGGCGGCCTA
TTGATACACTGCTCGAGTGATTTTAACTTTTATCTGATCATTCAAACGCATAAATTAGTCTTGAGAACTT
CAATTCATTTGATACTCCAGTTAACATGCTATTTACATGCTCATTTAAATGGTAGTAGTGATTTATAAGC
CCACTTCCAGATGGAACTTACCTATACCAACGTGTTACTTATCGTTCTTAAGCCAACTTAATAGCATTCT
AAAATATATATGTATCTTTTGGCGGACTTATCTTCTTGTTGTTCTCGCATTCCAAAATCTCTATGTACAT
GCAAACTTTTATTGTCATAACTCGGGACTTTGCAGACTTTGAGGCCTATTTAATAGAGCTATAATCTTAC
AACAAAAAAAAACTAAAAGAGCTTTTTAAGCAATAAAAATATTCTGAAAAATTACAAATTAACAAAAAAT
TACCCAATGAAGCCTGCAAATTTGAAATCTTTAAGATCCTAGATATGCCAAGATGCACCCTAAAGTCCTT
AACTCATCTCCTTGGCTCGTTTCTAATCCCCCCTCTCGAGGGATCGAGACGATCGCATCGGGTCGGTCTT
TAAGTTTGGATGATCCATAAACTGTTGGTTTCTCCGTCCTCAGCGTCTAGACTTCATTAGCCGTGTAATG
TTGCGGAATTTATGTGGCAGGCACATTAAAATAACACCGATACACACTCTCATGGACGCGAACGTGTGTA
CAAGTATAGAGATATCGGGCCTAGGCGAAAAATGAAATTAAAAAAAAAAAAAAAACTGGCACCGGGAGGG
GCTTATTTTTCGGTGGTCGGGGATGCGGGGGACTTTGACCATAAAATACATGCTCCCAAAAAGCTCGCAC
ACTGCAAGAGATGCGGGGCACTTCTGAGTCCCATATTCATATGCACAAATGTGCATTGCTGGCATTATCA
GTAGAATGCAATTTCGGGAAATTTTCCATCGCATCACGAGACAATGAACGTAAGAGAGAAATGGAGCCTC
AAAGAGGGAGGGAGAGAGAGAGCTTGAGTGAACGAGCGAGCGACAATCGCGAGATAACGGCTGCCTTATC
AGCAATGCCGACCGCCCCATCACCCCACCCAAAACGCCCAACCACCACCCACCGCCGCCGTTCCCTTTCC
TCCATCGTCGAGAATTTCGAGTTCAGAGCAGCGCGACCGAAATGAAAAGAAACAATTTAAATTCCAAATG
TATAAAATAGGTAAACTATGGCTTTTATTTATTAATATTGACGGGGGCACAAAGGCGGTCACCTCAATAG
TGAATAACATGTTTTTTATAATGAATACTTTTCAAATTGTTATTAATATGCAATGACGTCTTAAATGTTT
CACTGCAGCTGAACTTTATTCTTTTCATTAAAACAGTCACCCGTTATTAAAAATAAATAGATTAAGTTTT
ATATTATTAAATTTGTAAGTATTGAAACAATTCCTTTTTTATTTTATATGAATTATCATTTAGTTGGGGT
TAATATCCCTTAAAGAGAGAAATTTGTATGCTTTAAGATTTAAAATATCTATTGCATTTATAGCTATAGC
TATAACTTCTCTTATTTCACGCAGAAAATACTCAAATAAAACATATCGATTTGGCATACCCCACTAATTT
TTTGGCCCCAAGTGTGTGAGAGTGTGTGAGGCGAAGCGCGCCACAAACATAAAAAAGCGGTGAAGTGAGC
GGTTGTGGAACGTGAGTGGATGCTAAGAGCAAGCTCTCACATACGCGGACATAGGTCGCACACACACACG
CACAGACCGCCTTTTTGCGCCGCCGAAACGAACACTTTTACGAAGGCGACGGCGAATCAGTTTCAGTTGT
CAGTTCGCATCCAACTAGAAAGCAGTTAACGAGTAGTCTGTGTTTTTTCGCTTGCGGTTAAAAGCCACGA
GGTCGTTCATCGTTCATCGTTTTCCTTTTCAACTTCAAGCAAAGCAAATATAAACCAATGCAAAAAACGC
AGTGATCTTTTGAGGCCCAAATCGTTTGGGGCCGAACACCGTTGATTCTAAAACGCAAATGTAGAAACAA
ATCAAGAAAGTGGAAAATAAATATGTTTCGCTTTCAAAACATGTGAATGTGCCGAACTCAAAACTGAAAC
GTAGAAGGAACGCGTTCGTTTTTTACATACGACAATCGTATAAAATAAGAGAAAAGCTCCAAAACGTATT
AAATAGCGATGCTTGGATGATCTTCGTAGCAGTCACGTTGTACATACAAATACATACATATGTACCTACT
ATATGGCACATAAAATACGTTACGCACACTAGTGGCGAATAAAAAGCGAATTGGAGCAATCTTACAAAAT
GGACAAGAAGTACTCCATTGGGCTCGCTATCGGCACAAACAGCGTCGGCTGGGCCGTCATTACGGACGAG
TACAAGGTGCCGAGCAAAAAATTCAAAGTTCTGGGCAATACCGATCGCCACAGCATAAAGAAGAACCTCA
TTGGCGCCCTCCTGTTCGACTCCGGGGAGACGGCCGAAGCCACGCGGCTCAAAAGAACAGCACGGCGCAG
ATATACCCGCAGAAAGAATCGGATCTGCTACCTGCAGGAGATCTTTAGTAATGAGATGGCTAAGGTGGAT
GACTCTTTCTTCCATAGGCTGGAGGAGTCCTTTTTGGTGGAGGAGGATAAAAAGCACGAGCGCCACCCAA
TCTTTGGCAATATCGTGGACGAGGTGGCGTACCATGAAAAGTACCCAACCATATATCATCTGAGGAAGAA
GCTTGTAGACAGTACTGATAAGGCTGACTTGCGGTTGATCTATCTCGCGCTGGCGCATATGATCAAATTT
CGGGGACACTTCCTCATCGAGGGGGACCTGAACCCAGACAACAGCGATGTCGATAAACTCTTTATCCAAC
TGGTTCAGACTTACAATCAGCTTTTCGAAGAGAACCCGATCAACGCATCCGGAGTTGACGCCAAAGCAAT
CCTGAGCGCTAGGCTGTCCAAATCCCGGCGGCTCGAAAACCTCATCGCACAGCTCCCTGGGGAGAAGAAG
AACGGCCTGTTTGGTAATCTTATCGCCCTGTCACTCGGGCTGACCCCCAACTTTAAATCTAACTTCGACC
TGGCCGAAGATGCCAAGCTTCAACTGAGCAAAGACACCTACGATGATGATCTCGACAATCTGCTGGCCCA
GATCGGCGACCAGTACGCAGACCTTTTTTTGGCGGCAAAGAACCTGTCAGACGCCATTCTGCTGAGTGAT
ATTCTGCGAGTGAACACGGAGATCACCAAAGCTCCGCTGAGCGCTAGTATGATCAAGCGCTATGATGAGC
ACCACCAAGACTTGACTTTGCTGAAGGCCCTTGTCAGACAGCAACTGCCTGAGAAGTACAAGGAAATTTT
CTTCGATCAGTCTAAAAATGGCTACGCCGGATACATTGACGGCGGAGCAAGCCAGGAGGAATTTTACAAA
TTTATTAAGCCCATCTTGGAAAAAATGGACGGCACCGAGGAGCTGCTGGTAAAGCTTAACAGAGAAGATC
TGTTGCGCAAACAGCGCACTTTCGACAATGGAAGCATCCCCCACCAGATTCACCTGGGCGAACTGCACGC
TATCCTCAGGCGGCAAGAGGATTTCTACCCCTTTTTGAAAGATAACAGGGAAAAGATTGAGAAAATCCTC
ACATTTCGGATACCCTACTATGTAGGCCCCCTCGCCCGGGGAAATTCCAGATTCGCGTGGATGACTCGCA
AATCAGAAGAGACCATCACTCCCTGGAACTTCGAGGAAGTCGTGGATAAGGGGGCCTCTGCCCAGTCCTT
CATCGAAAGGATGACTAACTTTGATAAAAATCTGCCTAACGAAAAGGTGCTTCCTAAACACTCTCTGCTG
TACGAGTACTTCACAGTTTATAACGAGCTCACCAAGGTCAAATACGTCACAGAAGGGATGAGAAAGCCAG
CATTCCTGTCTGGAGAGCAGAAGAAAGCTATCGTGGACCTCCTCTTCAAGACGAACCGGAAAGTTACCGT
GAAACAGCTCAAAGAAGACTATTTCAAAAAGATTGAATGTTTCGACTCTGTTGAAATCAGCGGAGTGGAG
GATCGCTTCAACGCATCCCTGGGAACGTATCACGATCTCCTGAAAATCATTAAAGACAAGGACTTCCTGG
ACAATGAGGAGAACGAGGACATTCTTGAGGACATTGTCCTCACCCTTACGTTGTTTGAAGATAGGGAGAT
GATTGAAGAACGCTTGAAAACTTACGCTCATCTCTTCGACGACAAAGTCATGAAACAGCTCAAGAGGCGC
CGATATACAGGATGGGGGCGGCTGTCAAGAAAACTGATCAATGGGATCCGAGACAAGCAGAGTGGAAAGA
CAATCCTGGATTTTCTTAAGTCCGATGGATTTGCCAACCGGAACTTCATGCAGTTGATCCATGATGACTC
TCTCACCTTTAAGGAGGACATCCAGAAAGCACAAGTTTCTGGCCAGGGGGACAGTCTTCACGAGCACATC
GCTAATCTTGCAGGTAGCCCAGCTATCAAAAAGGGAATACTGCAGACCGTTAAGGTCGTGGATGAACTCG
TCAAAGTAATGGGAAGGCATAAGCCCGAGAATATCGTTATCGAGATGGCCCGAGAGAACCAAACTACCCA
GAAGGGACAGAAGAACAGTAGGGAAAGGATGAAGAGGATTGAAGAGGGTATAAAAGAACTGGGGTCCCAA
ATCCTTAAGGAACACCCAGTTGAAAACACCCAGCTTCAGAATGAGAAGCTCTACCTGTACTACCTGCAGA
ACGGCAGGGACATGTACGTGGATCAGGAACTGGACATCAATCGGCTCTCCGACTACGACGTGGCTGCTAT
CGTGCCCCAGTCTTTTCTCAAAGATGATTCTATTGATAATAAAGTGTTGACAAGATCCGATAAAGCTAGA
GGGAAGAGTGATAACGTCCCCTCAGAAGAAGTTGTCAAGAAAATGAAAAATTATTGGCGGCAGCTGCTGA
ACGCCAAACTGATCACACAACGGAAGTTCGATAATCTGACTAAGGCTGAACGAGGTGGCCTGTCTGAGTT
GGATAAAGCCGGCTTCATCAAAAGGCAGCTTGTTGAGACACGCCAGATCACCAAGCACGTGGCCCAAATT
CTCGATTCACGCATGAACACCAAGTACGATGAAAATGACAAACTGATTCGAGAGGTGAAAGTTATTACTC
TGAAGTCTAAGCTGGTCTCAGATTTCAGAAAGGACTTTCAGTTTTATAAGGTGAGAGAGATCAACAATTA
CCACCATGCGCATGATGCCTACCTGAATGCAGTGGTAGGCACTGCACTTATCAAAAAATATCCCAAGCTT
GAATCTGAATTTGTTTACGGAGACTATAAAGTGTACGATGTTAGGAAAATGATCGCAAAGTCTGAGCAGG
AAATAGGCAAGGCCACCGCTAAGTACTTCTTTTACAGCAATATTATGAATTTTTTCAAGACCGAGATTAC
ACTGGCCAATGGAGAGATTCGGAAGCGACCACTTATCGAAACAAACGGAGAAACAGGAGAAATCGTGTGG
GACAAGGGTAGGGATTTCGCGACAGTCCGGAAGGTCCTGTCCATGCCGCAGGTGAACATCGTTAAAAAGA
CCGAAGTACAGACCGGAGGCTTCTCCAAGGAAAGTATCCTCCCGAAAAGGAACAGCGACAAGCTGATCGC
ACGCAAAAAAGATTGGGACCCCAAGAAATACGGCGGATTCGATTCTCCTACAGTCGCTTACAGTGTACTG
GTTGTGGCCAAAGTGGAGAAAGGGAAGTCTAAAAAACTCAAAAGCGTCAAGGAACTGCTGGGCATCACAA
TCATGGAGCGATCAAGCTTCGAAAAAAACCCCATCGACTTTCTCGAGGCGAAAGGATATAAAGAGGTCAA
AAAAGACCTCATCATTAAGCTTCCCAAGTACTCTCTCTTTGAGCTTGAAAACGGCCGGAAACGAATGCTC
GCTAGTGCGGGCGAGCTGCAGAAAGGTAACGAGCTGGCACTGCCCTCTAAATACGTTAATTTCTTGTATC
TGGCCAGCCACTATGAAAAGCTCAAAGGGTCTCCCGAAGATAATGAGCAGAAGCAGCTGTTCGTGGAACA
ACACAAACACTACCTTGATGAGATCATCGAGCAAATAAGCGAATTCTCCAAAAGAGTGATCCTCGCCGAC
GCTAACCTCGATAAGGTGCTTTCTGCTTACAATAAGCACAGGGATAAGCCCATCAGGGAGCAGGCAGAAA
ACATTATCCACTTGTTTACTCTGACCAACTTGGGCGCGCCTGCAGCCTTCAAGTACTTCGACACCACCAT
AGACAGAAAGCGGTACACCTCTACAAAGGAGGTCCTGGACGCCACACTGATTCATCAGTCAATTACGGGG
CTCTATGAAACAAGAATCGACCTCTCTCAGCTCGGTGGAGACAGCAGGGCTGACCCCAAGAAGAAGAGGA
AGGTGGAGGCCAGCGGTTCCGGACGGGCTGACGCATTGGACGATTTTGATCTGGATATGCTGGGAAGTGA
CGCCCTCGATGATTTTGACCTTGACATGCTTGGTTCGGATGCCCTTGATGACTTTGACCTCGACATGCTC
GGCAGTGACGCCCTTGATGATTTCGACCTGGACATGCTGATTAACTCTAGAAGTTCCGGATCTCCGAAAA
AGAAACGCAAAGTTGGTAGCCAGTACCTGCCCGACACCGACGACCGGCACCGGATCGAGGAAAAGCGGAA
GCGGACCTACGAGACATTCAAGAGCATCATGAAGAAGTCCCCCTTCAGCGGCCCCACCGACCCTAGACCT
CCACCTAGAAGAATCGCCGTGCCCAGCAGATCCAGCGCCAGCGTGCCAAAACCTGCCCCCCAGCCTTACC
CCTTCACCAGCAGCCTGAGCACCATCAACTACGACGAGTTCCCTACCATGGTGTTCCCCAGCGGCCAGAT
CTCTCAGGCCTCTGCTCTGGCTCCAGCCCCTCCTCAGGTGCTGCCTCAGGCTCCTGCTCCTGCACCAGCT
CCAGCCATGGTGTCTGCACTGGCTCAGGCACCAGCACCCGTGCCTGTGCTGGCTCCTGGACCTCCACAGG
CTGTGGCTCCACCAGCCCCTAAACCTACACAGGCCGGCGAGGGCACACTGTCTGAAGCTCTGCTGCAGCT
GCAGTTCGACGACGAGGATCTGGGAGCCCTGCTGGGAAACAGCACCGATCCTGCCGTGTTCACCGACCTG
GCCAGCGTGGACAACAGCGAGTTCCAGCAGCTGCTGAACCAGGGCATCCCTGTGGCCCCTCACACCACCG
AGCCCATGCTCATGGAATACCCCGAGGCCATCACCCGGCTCCTGACAGGCGCTCAGAGGCCTCCTGATCC
AGCTCCTGCCCCTCTGGGAGCACCAGGCCTGCCTAATGGACTGCTGTCTGGCGACGAGGACTTCAGCTCT
ATCGCCGATATGGATTTCTCAGCCTTGCTGGGCTCTGGCAGCGGCAGCCGGGATTCCAGGGAAGGGATGT
TTTTGCCGAAGCCTGAGGCCGGCTCCGCTATTAGTGACGTGTTTGAGGGCCGCGAGGTGTGCCAGCCAAA
ACGAATCCGGCCATTTCATCCTCCAGGAAGTCCATGGGCCAACCGCCCACTCCCCGCCAGCCTCGCACCA
ACACCAACCGGTCCAGTACATGAGCCAGTCGGGTCACTGACCCCGGCACCAGTCCCTCAGCCACTGGATC
CAGCGCCCGCAGTGACTCCCGAGGCCAGTCACCTGTTGGAGGATCCCGATGAAGAGACGAGCCAGGCTGT
CAAAGCCCTTCGGGAGATGGCCGATACTGTGATTCCCCAGAAGGAAGAGGCTGCAATCTGTGGCCAAATG
GACCTTTCCCATCCGCCCCCAAGGGGCCATCTGGATGAGCTGACAACCACACTTGAGTCCATGACCGAGG
ATCTGAACCTGGACTCACCCCTGACCCCGGAATTGAACGAGATTCTGGATACCTTCCTGAACGACGAGTG
CCTCTTGCATGCCATGCATATCAGCACAGGACTGTCCATCTTCGACACATCTCTGTTTTGACCGACTCTA
GATCATAATCAGCCATACCACATTTGTAGAGGTTTTACTTGCTTTAAAAAACCTCCCACACCTCCCCCTG
AACCTGAAACATAAAATGAATGCAATTGTTGTTGTTAACTTGTTTATTGCAGCTTATAATGGTTACAAAT
AAAGCAATAGCATCACAAATTTCACAAATAAAGCATTTTTTTCACTGCATTCTAGTTGTGGTTTGCCCAA
ACTCATCAATGTATCTTAGCGGCTCGAGGGTACCTCTAGAGATCCACTAGTGTCGACGATGTAGGTCACG
GTCTCGAAGCCGCGGTGCGGGTGCCAGGGCGTGCCCTTGGGCTCCCCGGGCGCGTACTCCACCTCACCCA
TCTGGTCCATCATGATGAACGGGTCGAGGTGGCGGTAGTTGATCCCGGCGAACGCGCGGCGCACCGGGAA
GCCCTCGCCCTCGAAACCGCTGGGCGCGGTGGTCACGGTGAGCACGGGACGTGCGACGGCGTCGGCGGGT
GCGGATACGCGGGGCAGCGTCAGCGGGTTCTCGACGGTCACGGCGGGCATGTCGACACTA
SEQ ID NO: 3-pAH-1
CTGACGCGCCCTGTAGCGGCGCATTAAGCGCGGCGGGTGTGGTGGTTACGCGCAGCGTGACCGCTACACT
TGCCAGCGCCCTAGCGCCCGCTCCTTTCGCTTTCTTCCCTTCCTTTCTCGCCACGTTCGCCGGCTTTCCC
CGTCAAGCTCTAAATCGGGGGCTCCCTTTAGGGTTCCGATTTAGTGCTTTACGGCACCTCGACCCCAAAA
AACTTGATTAGGGTGATGGTTCACGTAGTGGGCCATCGCCCTGATAGACGGTTTTTCGCCCTTTGACGTT
GGAGTCCACGTTCTTTAATAGTGGACTCTTGTTCCAAACTGGAACAACACTCAACCCTATCTCGGTCTAT
TCTTTTGATTTATAAGGGATTTTGCCGATTTCGGCCTATTGGTTAAAAAATGAGCTGATTTAACAAAAAT
TTAACGCGAATTTTAACAAAATATTAACGCTTACAATTTCCATTCGCCATTCAGGCTGCGCAACTGTTGG
GAAGGGCGATCGGTGCGGGCCTCTTCGCTATTACGCCAGCTGGCGAAAGGGGGATGTGCTGCAAGGCGAT
TAAGTTGGGTAACGCCAGGGTTTTCCCAGTCACGACGTTGTAAAACGACGGCCAGTGAATTGTAATACGA
CTCACTATAGGGCGAATTGGGTACGTACCGGGCCCCTAGTATGTATGTAAGTTAATAAAACCCATTTTTG
CGGAAAGTAGATAAAAAAAACATTTTTTTTTTTTACTGCACTGGATATCATTGAACTTATCTGATCAGTT
TTAAATTTACTTCGATCCAAGGGTATTTGATGTACCAGGTTCTTTCGATTACCTCTCACTCAAAATGACA
TTCCACTCAAAGTCAGCGCTGTTTGCCTCCTTCTCTGTCCACAGAAATATCGCCGTCTCTTTCGCCGCTG
CGTCCGCTATCTCTTTCGCCACCGTTTGTAGCGTTACGTAGCGTCAATGTCCGCCTTCAGTTGCATTTTG
TCAGCGGTTTCGTGACGAAGCTCCAAGCGGTTTACGCCATCAATTAAACACAAAGTGCTGTGCCAAAACT
CCTCTCGCTTCTTATTTTTGTTTGTTTTTTGAGTGATTGGGGTGGTGATTGGTTTTGGGTGGGTAAGCAG
GGGAAAGTGTGAAAAATCCCGGCAATGGGCCAAGAGGATCAGGAGCTATTAATTCGCGGAGGCAGCAAAC
ACCCATCTGCCGAGCATCTGAACAATGTGAGTAGTACATGTGCATACATCTTAAGTTCACTTGATCTATA
GGAACTGCGATTGCAACATCAAATTGTCTGCGGCGTGAGAACTGCGACCCACAAAAATCCCAAACCGCAA
TTGCACAAACAAATAGTGACACGAAACAGATTATTCTGGTAGCTGTTCTCGCTATATAAGACAATTTTTG
AGATCATATCATGATCAAGACATCTAAAGGCATTCATTTTCGACTATATTCTTTTTTACAAAAAATATAA
CAACCAGATATTTTAAGCTGATCCTAGATGCACAAAAAATAAATAAAAGTATAAACCTACTTCGTAGGAT
ACTTCGGGGTACTTTTTGTTCGGGGTTAGATGAGCATAACGCTTGTAGTTGATATTTGAGATCCCCTATC
ATTGCAGGGTGACAGCGGAGCGGCTTCGCAGAGCTGCATTAACCAGGGCTTCGGGCAGGCCAAAAACTAC
GGCACGCTCCGGCCACCCAGTCCGCCGGAGGACTCCGGTTCAGGGAGCGGCCAACTAGCCGAGAACCTCA
CCTATGCCTGGCACAATATGGACATCTTTGGGGCGGTCAATCAGCCGGGCTCCGGATGGCGGCAGCTGGT
CAACCGGACACGCGGACTATTCTGCAACGAGCGACACATACCGGCGCCCAGGAAACATTTGCTCAAGAAC
GGTGAGTTTCTATTCGCAGTCGGCTGATCTGTGTGAAATCTTAATAAAGGGTCCAATTACCAATTTGAAA
CTCAGTTTGCGGCGTGGCCTATCCGGGCGAACTTTTGGCCGTGATGGGCAGTTCCGGTGCCGGAAAGACG
ACCCTGCTGAATGCCCTTGCCTTTCGATCGCCGCAGGGCATCCAAGTATCGCCATCCGGGATGCGACTGC
TCAATGGCCAACCTGTGGACGCCAAGGAGATGCAGGCCAGGTGCGCCTATGTCCAGCAGGATGACCTCTT
TATCGGCTCCCTAACGGCCAGGGAACACCTGATTTTCCAGGCCATGGTGCGGATGCCACGACATCTGACC
TATCGGCAGCGAGTGGCCCGCGTGGATCAGGTGATCCAGGAGCTTTCGCTCAGCAAATGTCAGCACACGA
TCATCGGTGTGCCCGGCAGGGTGAAAGGTCTGTCCGGCGGAGAAAGGAAGCGTCTGGCATTCGCCTCCGA
GGCACTAACCGATCCGCCGCTTCTGATCTGCGATGAGCCCACCTCCGGACTGGACTCATTTACCGCCCAC
AGCGTCGTCCAGGTGCTGAAGAAGCTGTCGCAGAAGGGCAAGACCGTCATCCTGACCATTCATCAGCCGT
CTTCCGAGCTGTTTGAGCTCTTTGACAAGATCCTTCTGATGGCCGAGGGCAGGGTAGCTTTCTTGGGCAC
TCCCAGCGAAGCCGTCGACTTCTTTTCCTAGTGAGTTCGATGTGTTTATTAAGGGTATCTAGCATTACAT
TACATCTCAACTCCTATCCAGCGTGGGTGCCCAGTGTCCTACCAACTACAATCCGGCGGACTTTTACGTA
CAGGTGTTGGCCGTTGTGCCCGGACGGGAGATCGAGTCCCGTGATCGGATCGCCAAGATATGCGACAATT
TTGCTATTAGCAAAGTAGCCCGGGATATGGAGCAGTTGTTGGCCACCAAAAATTTGGAGAAGCCACTGGA
GCAGCCGGAGAATGGGTACACCTACAAGGCCACCTGGTTCATGCAGTTCCGGGCGGTCCTGTGGCGATCC
TGGCTGTCGGTGCTCAAGGAACCACTCCTCGTAAAAGTGCGACTTATTCAGACAACGGTGAGTGGTTCCA
GTGGAAACAAATGATATAACGCTTACAATTCTTGGAAACAAATTCGCTAGATTTTAGTTAGAATTGCCTG
ATTCCACACCCTTCTTAGTTTTTTTCAATGAGATGTATAGTTTATAGTTTTGCAGAAAATAAATAAATTT
CATTTAACTCGCGAACATGTTGAAGATATGAATATTAATGAGATGCGAGTAACATTTTAATTTGCAGATG
GTTGCCATCTTGATTGGCCTCATCTTTTTGGGCCAACAACTCACGCAAGTGGGCGTGATGAATATCAACG
GAGCCATCTTCCTCTTCCTGACCAACATGACCTTTCAAAACGTCTTTGCCACGATAAATGTAAGTCTTGT
TTAGAATACATTTGCATATTAATAATTTACTAACTTTCTAATGAATCGATTCGATTTAGGTGTTCACCTC
AGAGCTGCCAGTTTTTATGAGGGAGGCCCGAAGTCGACTTTATCGCTGTGACACATACTTTCTGGGCAAA
ACGATTGCCGAATTACCGCTTTTTCTCACAGTGCCACTGGTCTTCACGGCGATTGCCTATCCGATGATCG
GACTGCGGGCCGGAGTGCTGCACTTCTTCAACTGCCTGGCGCTGGTCACTCTGGTGGCCAATGTGTCAAC
GTCCTTCGGATATCTAATATCCTGCGCCAGCTCCTCGACCTCGATGGCGCTGTCTGTGGGTCCGCCGGTT
ATCATACCATTCCTGCTCTTTGGCGGCTTCTTCTTGAACTCGGGCTCGGTGCCAGTATACCTCAAATGGT
TGTCGTACCTCTCATGGTTCCGTTACGCCAACGAGGGTCTGCTGATTAACCAATGGGCGGACGTGGAGCC
GGGCGAAATTAGCTGCACATCGTCGAACACCACGTGCCCCAGTTCGGGCAAGGTCATCCTGGAGACGCTT
AACTTCTCCGCCGCCGATCTGCCGCTGGACTACGTGGGTCTGGCCATTCTCATCGTGAGCTTCCGGGTGC
TCGCATATCTGGCTCTAAGACTTCGGGCCCGACGCAAGGAGTAGCCGACATATATCCGAAATAACTGCTT
GTTTTTTTTTTTTACCATTATTACCATCGTGTTTACTGTTTATTGCCCCCTCAAAAAGCTAATGTAATTA
TATTTGTGCCAATAAAAACAAGATATGACCTATAGAATACAAGTATTTCCCCTTCGAACATCCCCACAAG
TAGACTTTGGATTTGTCTTCTAACCAAAAGACTTACACACCTGCATACCTTACATCAAAAACTCGTTTAT
CGCTACATAAAACACCGGGATATATTTTTTATATACATACTTTTCAAATCGCGCGCCCTCTTCATAATTC
ACCTCCACCACACCACGTTTCGTAGTTGCTCTTTCGCTGTCTCCCACCCGCTCTCCGCAACACATTCACC
TTTTGTTCGACGACCTTGGAGCGACTGTCGTTAGTTCCGCGCGATTCGGTTCGCTCAAATGGTTCCGAGT
GGTTCATTTCGTCTCAATAGAAATTAGTAATAAATATTTGTATGTACAATTTATTTGCTCCAATATATTT
GTATATATTTCCCTCACAGCTATATTTATTCTAATTTAATATTATGACTTTTTAAGGTAATTTTTTGTGA
CCTGTTCGGAGTGATTAGCGTTACAATTTGAACTGAAAGTGACATCCAGTGTTTGTTCCTTGTGTAGATG
CATCTCAAAAAAATGGTGGGCATAATAGTGTTGTTTATATATATCAAAAATAACAACTATAATAATAAGA
ATACATTTAATTTAGAAAATGCTTGGATTTCACTGGAACTAGGCTAGCATAACTTCGTATAATGTATGCT
ATACGAAGTTATGCTAGCGGATCCGGGAATTGGGAATTCACGTAAGTACTGTCTGCAGCGTAAGCTTCGT
ACGTAGCGTCAAATTTGGTTGTGATTACGAGACGGAGACCGAGACGGCGACGACAGTTAGCCATTCGCCA
CGCGCCAACGCAAATGAAACGCTCTATACATATTTTTGTATATTTTCTGTTTTTTTTTGCCGCTGACAAT
TATGATCAAGTATTAGCTGGCGATAGCTGAAACGTCTGTGTAATTTCAATGGAATGGAATGGGAAGTGGG
CGGCCTATTGATACACTGCTCGAGTGATTTTAACTTTTATCTGATCATTCAAACGCATAAATTAGTCTTG
AGAACTTCAATTCATTTGATACTCCAGTTAACATGCTATTTACATGCTCATTTAAATGGTAGTAGTGATT
TATAAGCCCACTTCCAGATGGAACTTACCTATACCAACGTGTTACTTATCGTTCTTAAGCCAACTTAATA
GCATTCTAAAATATATATGTATCTTTTGGCGGACTTATCTTCTTGTTGTTCTCGCATTCCAAAATCTCTA
TGTACATGCAAACTTTTATTGTCATAACTCGGGACTTTGCAGACTTTGAGGCCTATTTAATAGAGCTATA
ATCTTACAACAAAAAAAAACTAAAAGAGCTTTTTAAGCAATAAAAATATTCTGAAAAATTACAAATTAAC
AAAAAATTACCCAATGAAGCCTGCAAATTTGAAATCTTTAAGATCCTAGATATGCCAAGATGCACCCTAA
AGTCCTTAACTCATCTCCTTGGCTCGTTTCTAATCCCCCCTCTCGAGGGATCGAGACGATCGCATCGGGT
CGGTCTTTAAGTTTGGATGATCCATAAACTGTTGGTTTCTCCGTCCTCAGCGTCTAGACTTCATTAGCCG
TGTAATGTTGCGGAATTTATGTGGCAGGCACATTAAAATAACACCGATACACACTCTCATGGACGCGAAC
GTGTGTACAAGTATAGAGATATCGGGCCTAGGCGAAAAATGAAATTAAAAAAAAAAAAAAAACTGGCACC
GGGAGGGGCTTATTTTTCGGTGGTCGGGGATGCGGGGGACTTTGACCATAAAATACATGCTCCCAAAAAG
CTCGCACACTGCAAGAGATGCGGGGCACTTCTGAGTCCCATATTCATATGCACAAATGTGCATTGCTGGC
ATTATCAGTAGAATGCAATTTCGGGAAATTTTCCATCGCATCACGAGACAATGAACGTAAGAGAGAAATG
GAGCCTCAAAGAGGGAGGGAGAGAGAGAGCTTGAGTGAACGAGCGAGCGACAATCGCGAGATAACGGCTG
CCTTATCAGCAATGCCGACCGCCCCATCACCCCACCCAAAACGCCCAACCACCACCCACCGCCGCCGTTC
CCTTTCCTCCATCGTCGAGAATTTCGAGTTCAGAGCAGCGCGACCGAAATGAAAAGAAACAATTTAAATT
CCAAATGTATAAAATAGGTAAACTATGGCTTTTATTTATTAATATTGACGGGGGCACAAAGGCGGTCACC
TCAATAGTGAATAACATGTTTTTTATAATGAATACTTTTCAAATTGTTATTAATATGCAATGACGTCTTA
AATGTTTCACTGCAGCTGAACTTTATTCTTTTCATTAAAACAGTCACCCGTTATTAAAAATAAATAGATT
AAGTTTTATATTATTAAATTTGTAAGTATTGAAACAATTCCTTTTTTATTTTATATGAATTATCATTTAG
TTGGGGTTAATATCCCTTAAAGAGAGAAATTTGTATGCTTTAAGATTTAAAATATCTATTGCATTTATAG
CTATAGCTATAACTTCTCTTATTTCACGCAGAAAATACTCAAATAAAACATATCGATTTGGCATACCCCA
CTAATTTTTTGGCCCCAAGTGTGTGAGAGTGTGTGAGGCGAAGCGCGCCACAAACATAAAAAAGCGGTGA
AGTGAGCGGTTGTGGAACGTGAGTGGATGCTAAGAGCAAGCTCTCACATACGCGGACATAGGTCGCACAC
ACACACGCACAGACCGCCTTTTTGCGCCGCCGAAACGAACACTTTTACGAAGGCGACGGCGAATCAGTTT
CAGTTGTCAGTTCGCATCCAACTAGAAAGCAGTTAACGAGTAGTCTGTGTTTTTTCGCTTGCGGTTAAAA
GCCACGAGGTCGTTCATCGTTCATCGTTTTCCTTTTCAACTTCAAGCAAAGCAAATATAAACCAATGCAA
AAAACGCAGTGATCTTTTGAGGCCCAAATCGTTTGGGGCCGAACACCGTTGATTCTAAAACGCAAATGTA
GAAACAAATCAAGAAAGTGGAAAATAAATATGTTTCGCTTTCAAAACATGTGAATGTGCCGAACTCAAAA
CTGAAACGTAGAAGGAACGCGTTCGTTTTTTACATACGACAATCGTATAAAATAAGAGAAAAGCTCCAAA
ACGTATTAAATAGCGATGCTTGGATGATCTTCGTAGCAGTCACGTTGTACATACAAATACATACATATGT
ACCTACTATATGGCACATAAAATACGTTACGCACACTAGTGGCGAATAAAAAGCGAATTGGAGCAATCTT
ACAAAATGGACAAGAAGTACTCCATTGGGCTCGCTATCGGCACAAACAGCGTCGGCTGGGCCGTCATTAC
GGACGAGTACAAGGTGCCGAGCAAAAAATTCAAAGTTCTGGGCAATACCGATCGCCACAGCATAAAGAAG
AACCTCATTGGCGCCCTCCTGTTCGACTCCGGGGAGACGGCCGAAGCCACGCGGCTCAAAAGAACAGCAC
GGCGCAGATATACCCGCAGAAAGAATCGGATCTGCTACCTGCAGGAGATCTTTAGTAATGAGATGGCTAA
GGTGGATGACTCTTTCTTCCATAGGCTGGAGGAGTCCTTTTTGGTGGAGGAGGATAAAAAGCACGAGCGC
CACCCAATCTTTGGCAATATCGTGGACGAGGTGGCGTACCATGAAAAGTACCCAACCATATATCATCTGA
GGAAGAAGCTTGTAGACAGTACTGATAAGGCTGACTTGCGGTTGATCTATCTCGCGCTGGCGCATATGAT
CAAATTTCGGGGACACTTCCTCATCGAGGGGGACCTGAACCCAGACAACAGCGATGTCGATAAACTCTTT
ATCCAACTGGTTCAGACTTACAATCAGCTTTTCGAAGAGAACCCGATCAACGCATCCGGAGTTGACGCCA
AAGCAATCCTGAGCGCTAGGCTGTCCAAATCCCGGCGGCTCGAAAACCTCATCGCACAGCTCCCTGGGGA
GAAGAAGAACGGCCTGTTTGGTAATCTTATCGCCCTGTCACTCGGGCTGACCCCCAACTTTAAATCTAAC
TTCGACCTGGCCGAAGATGCCAAGCTTCAACTGAGCAAAGACACCTACGATGATGATCTCGACAATCTGC
TGGCCCAGATCGGCGACCAGTACGCAGACCTTTTTTTGGCGGCAAAGAACCTGTCAGACGCCATTCTGCT
GAGTGATATTCTGCGAGTGAACACGGAGATCACCAAAGCTCCGCTGAGCGCTAGTATGATCAAGCGCTAT
GATGAGCACCACCAAGACTTGACTTTGCTGAAGGCCCTTGTCAGACAGCAACTGCCTGAGAAGTACAAGG
AAATTTTCTTCGATCAGTCTAAAAATGGCTACGCCGGATACATTGACGGCGGAGCAAGCCAGGAGGAATT
TTACAAATTTATTAAGCCCATCTTGGAAAAAATGGACGGCACCGAGGAGCTGCTGGTAAAGCTTAACAGA
GAAGATCTGTTGCGCAAACAGCGCACTTTCGACAATGGAAGCATCCCCCACCAGATTCACCTGGGCGAAC
TGCACGCTATCCTCAGGCGGCAAGAGGATTTCTACCCCTTTTTGAAAGATAACAGGGAAAAGATTGAGAA
AATCCTCACATTTCGGATACCCTACTATGTAGGCCCCCTCGCCCGGGGAAATTCCAGATTCGCGTGGATG
ACTCGCAAATCAGAAGAGACCATCACTCCCTGGAACTTCGAGGAAGTCGTGGATAAGGGGGCCTCTGCCC
AGTCCTTCATCGAAAGGATGACTAACTTTGATAAAAATCTGCCTAACGAAAAGGTGCTTCCTAAACACTC
TCTGCTGTACGAGTACTTCACAGTTTATAACGAGCTCACCAAGGTCAAATACGTCACAGAAGGGATGAGA
AAGCCAGCATTCCTGTCTGGAGAGCAGAAGAAAGCTATCGTGGACCTCCTCTTCAAGACGAACCGGAAAG
TTACCGTGAAACAGCTCAAAGAAGACTATTTCAAAAAGATTGAATGTTTCGACTCTGTTGAAATCAGCGG
AGTGGAGGATCGCTTCAACGCATCCCTGGGAACGTATCACGATCTCCTGAAAATCATTAAAGACAAGGAC
TTCCTGGACAATGAGGAGAACGAGGACATTCTTGAGGACATTGTCCTCACCCTTACGTTGTTTGAAGATA
GGGAGATGATTGAAGAACGCTTGAAAACTTACGCTCATCTCTTCGACGACAAAGTCATGAAACAGCTCAA
GAGGCGCCGATATACAGGATGGGGGCGGCTGTCAAGAAAACTGATCAATGGGATCCGAGACAAGCAGAGT
GGAAAGACAATCCTGGATTTTCTTAAGTCCGATGGATTTGCCAACCGGAACTTCATGCAGTTGATCCATG
ATGACTCTCTCACCTTTAAGGAGGACATCCAGAAAGCACAAGTTTCTGGCCAGGGGGACAGTCTTCACGA
GCACATCGCTAATCTTGCAGGTAGCCCAGCTATCAAAAAGGGAATACTGCAGACCGTTAAGGTCGTGGAT
GAACTCGTCAAAGTAATGGGAAGGCATAAGCCCGAGAATATCGTTATCGAGATGGCCCGAGAGAACCAAA
CTACCCAGAAGGGACAGAAGAACAGTAGGGAAAGGATGAAGAGGATTGAAGAGGGTATAAAAGAACTGGG
GTCCCAAATCCTTAAGGAACACCCAGTTGAAAACACCCAGCTTCAGAATGAGAAGCTCTACCTGTACTAC
CTGCAGAACGGCAGGGACATGTACGTGGATCAGGAACTGGACATCAATCGGCTCTCCGACTACGACGTGG
CTGCTATCGTGCCCCAGTCTTTTCTCAAAGATGATTCTATTGATAATAAAGTGTTGACAAGATCCGATAA
AGCTAGAGGGAAGAGTGATAACGTCCCCTCAGAAGAAGTTGTCAAGAAAATGAAAAATTATTGGCGGCAG
CTGCTGAACGCCAAACTGATCACACAACGGAAGTTCGATAATCTGACTAAGGCTGAACGAGGTGGCCTGT
CTGAGTTGGATAAAGCCGGCTTCATCAAAAGGCAGCTTGTTGAGACACGCCAGATCACCAAGCACGTGGC
CCAAATTCTCGATTCACGCATGAACACCAAGTACGATGAAAATGACAAACTGATTCGAGAGGTGAAAGTT
ATTACTCTGAAGTCTAAGCTGGTCTCAGATTTCAGAAAGGACTTTCAGTTTTATAAGGTGAGAGAGATCA
ACAATTACCACCATGCGCATGATGCCTACCTGAATGCAGTGGTAGGCACTGCACTTATCAAAAAATATCC
CAAGCTTGAATCTGAATTTGTTTACGGAGACTATAAAGTGTACGATGTTAGGAAAATGATCGCAAAGTCT
GAGCAGGAAATAGGCAAGGCCACCGCTAAGTACTTCTTTTACAGCAATATTATGAATTTTTTCAAGACCG
AGATTACACTGGCCAATGGAGAGATTCGGAAGCGACCACTTATCGAAACAAACGGAGAAACAGGAGAAAT
CGTGTGGGACAAGGGTAGGGATTTCGCGACAGTCCGGAAGGTCCTGTCCATGCCGCAGGTGAACATCGTT
AAAAAGACCGAAGTACAGACCGGAGGCTTCTCCAAGGAAAGTATCCTCCCGAAAAGGAACAGCGACAAGC
TGATCGCACGCAAAAAAGATTGGGACCCCAAGAAATACGGCGGATTCGATTCTCCTACAGTCGCTTACAG
TGTACTGGTTGTGGCCAAAGTGGAGAAAGGGAAGTCTAAAAAACTCAAAAGCGTCAAGGAACTGCTGGGC
ATCACAATCATGGAGCGATCAAGCTTCGAAAAAAACCCCATCGACTTTCTCGAGGCGAAAGGATATAAAG
AGGTCAAAAAAGACCTCATCATTAAGCTTCCCAAGTACTCTCTCTTTGAGCTTGAAAACGGCCGGAAACG
AATGCTCGCTAGTGCGGGCGAGCTGCAGAAAGGTAACGAGCTGGCACTGCCCTCTAAATACGTTAATTTC
TTGTATCTGGCCAGCCACTATGAAAAGCTCAAAGGGTCTCCCGAAGATAATGAGCAGAAGCAGCTGTTCG
TGGAACAACACAAACACTACCTTGATGAGATCATCGAGCAAATAAGCGAATTCTCCAAAAGAGTGATCCT
CGCCGACGCTAACCTCGATAAGGTGCTTTCTGCTTACAATAAGCACAGGGATAAGCCCATCAGGGAGCAG
GCAGAAAACATTATCCACTTGTTTACTCTGACCAACTTGGGCGCGCCTGCAGCCTTCAAGTACTTCGACA
CCACCATAGACAGAAAGCGGTACACCTCTACAAAGGAGGTCCTGGACGCCACACTGATTCATCAGTCAAT
TACGGGGCTCTATGAAACAAGAATCGACCTCTCTCAGCTCGGTGGAGACAGCAGGGCTGACCCCAAGAAG
AAGAGGAAGGTGGAGGCCAGCGGTTCCGGACGGGCTGACGCATTGGACGATTTTGATCTGGATATGCTGG
GAAGTGACGCCCTCGATGATTTTGACCTTGACATGCTTGGTTCGGATGCCCTTGATGACTTTGACCTCGA
CATGCTCGGCAGTGACGCCCTTGATGATTTCGACCTGGACATGCTGATTAACTCTAGAAGTTCCGGATCT
CCGAAAAAGAAACGCAAAGTTGGTAGCCAGTACCTGCCCGACACCGACGACCGGCACCGGATCGAGGAAA
AGCGGAAGCGGACCTACGAGACATTCAAGAGCATCATGAAGAAGTCCCCCTTCAGCGGCCCCACCGACCC
TAGACCTCCACCTAGAAGAATCGCCGTGCCCAGCAGATCCAGCGCCAGCGTGCCAAAACCTGCCCCCCAG
CCTTACCCCTTCACCAGCAGCCTGAGCACCATCAACTACGACGAGTTCCCTACCATGGTGTTCCCCAGCG
GCCAGATCTCTCAGGCCTCTGCTCTGGCTCCAGCCCCTCCTCAGGTGCTGCCTCAGGCTCCTGCTCCTGC
ACCAGCTCCAGCCATGGTGTCTGCACTGGCTCAGGCACCAGCACCCGTGCCTGTGCTGGCTCCTGGACCT
CCACAGGCTGTGGCTCCACCAGCCCCTAAACCTACACAGGCCGGCGAGGGCACACTGTCTGAAGCTCTGC
TGCAGCTGCAGTTCGACGACGAGGATCTGGGAGCCCTGCTGGGAAACAGCACCGATCCTGCCGTGTTCAC
CGACCTGGCCAGCGTGGACAACAGCGAGTTCCAGCAGCTGCTGAACCAGGGCATCCCTGTGGCCCCTCAC
ACCACCGAGCCCATGCTGATGGAATACCCCGAGGCCATCACCCGGCTCGTGACAGGCGCTCAGAGGCCTC
CTGATCCAGCTCCTGCCCCTCTGGGAGCACCAGGCCTGCCTAATGGACTGCTGTCTGGCGACGAGGACTT
CAGCTCTATCGCCGATATGGATTTCTCAGCCTTGCTGGGCTCTGGCAGCGGCAGCCGGGATTCCAGGGAA
GGGATGTTTTTGCCGAAGCCTGAGGCCGGCTCCGCTATTAGTGACGTGTTTGAGGGCCGCGAGGTGTGCC
AGCCAAAACGAATCCGGCCATTTCATCCTCCAGGAAGTCCATGGGCCAACCGCCCACTCCCCGCCAGCCT
CGCACCAACACCAACCGGTCCAGTACATGAGCCAGTCGGGTCACTGACCCCGGCACCAGTCCCTCAGCCA
CTGGATCCAGCGCCCGCAGTGACTCCCGAGGCCAGTCACCTGTTGGAGGATCCCGATGAAGAGACGAGCC
AGGCTGTCAAAGCCCTTCGGGAGATGGCCGATACTGTGATTCCCCAGAAGGAAGAGGCTGCAATCTGTGG
CCAAATGGACCTTTCCCATCCGCCCCCAAGGGGCCATCTGGATGAGCTGACAACCACACTTGAGTCCATG
ACCGAGGATCTGAACCTGGACTCACCCCTGACCCCGGAATTGAACGAGATTCTGGATACCTTCCTGAACG
ACGAGTGCCTCTTGCATGCCATGCATATCAGCACAGGACTGTCCATCTTCGACACATCTCTGTTTTGACC
GACTCTAGATCATAATCAGCCATACCACATTTGTAGAGGTTTTACTTGCTTTAAAAAACCTCCCACACCT
CCCCCTGAACCTGAAACATAAAATGAATGCAATTGTTGTTGTTAACTTGTTTATTGCAGCTTATAATGGT
TACAAATAAAGCAATAGCATCACAAATTTCACAAATAAAGCATTTTTTTCACTGCATTCTAGTTGTGGTT
TGCCCAAACTCATCAATGTATCTTAGCGGCTCGAGGGTACAAGCCGAATTGATCCACTAGAAGGCCTAAT
TCGGTACACTAGTTGGCCACGTAATAAGTGTGCGTTGAATTTATTCGCAAAAACATTGCATATTTTCGGC
AAAGTAAAATTTTGTTGCATACCTTATCAAAAAATAAGTGCTGCATACTTTTTAGAGAAACCAAATAATT
TTTTATTGCATACCCGTTTTTAATAAAATACATTGCATACCCTCTTTTAATAAAAAATATTGCATACTTT
GACGAAACAAATTTTCGTTGCATACCCAATAAAAGATTATTATATTGCATACCCGTTTTTAATAAAATAC
ATTGCATACCCTCTTTTAATAAAGAATATTGCATACGTTGACGAAACAAATTTTCGTTGCATACCCAATA
AAAGATTATTATATTGCATACCTTTTCTTGCCATACCATTTAGCCGATCAATTGAGATCTGAATTCATTT
TCAACGTCCTCGATAGTATAGTGGTTAGTATCCCCGCCTGTCACGCGGGAGACCGGGGTTCAATTCCCCG
TCGGGGAGAATCTGTGATTCTTTTTTTTTTTCTTTTACTTTGTTATATAAACAATTTTTGTTTTAATTGA
ATCTAATTTGCCATTGCTTTTAGGAATCTCAGGCATCCAGCAAGCGTTTGTCCGCCGAATCGCCCATCAG
TGAAGAAGATCCTGTGGCGGCTACGAAAATCTCCCCGGCCATGTCGGCCTCCACCTCCAGCGAAAAACCC
ATCAGCGAGCTGGCCACCTCTGTGCTGACCCACCGCTTTCCAGACTCCACCTCCTCACCCGGCGAACATG
GCCTTGGACGAATGCAGTTGTCGATCCGCTACAGCGCCCAGCGTCAAAAACTAGACGTGACCATACACAA
AATCCAGAAGATACCACTTCGCGATCCCAGCAATATCCCCGATCCGTATGTTAAGCTGTATCTGTTGCCT
GGACGCACCAAGGAGTCGAAACGCAAGACGAGCGTGATCAAGGACAACTGCAACCCCGTCTACGATGCAT
CCTTTGAGTACCTGATTTCCATTGCCGAACTCAGGCAGACGGAACTGGAGGTGACGGTGTGCACCCAAAA
GGGATTCCTATCCGGCGGTAGTCCCATCATTGGCATGGTAGGTACCCGAAAGCAACCCCTTAGTTACAGA
CACAGCGCGTACGTCCTTCGCATCCTTATGATTCCCAAGTACATATTCTGCAAGAGTACAGTATATATAG
GAAAGATATCCGGGTGAACTTCGCGGCGGGGGCGGTTCGAAGCGTTTTAGAGCTAGAAATAGCAAGTTAA
AATAAGGCTAGTCCGTTATCAACTTGAAAAAGTGGCACCGAGTCGGTGCTTTTTTGCTCACCTGTGATTG
CTCCTACTCAAATACAAAAACATCAAATTTTCTGTCAATAAAGCATATTTATTTATATTTATTTTACAGG
AAAGAATTCCTTTTAAAGTGTATTTTAACCTATAATGAAAAACGATTAAAAAAAATACATAAAATAATTC
GAAAATTTTTGAATAGCCCAGGTTGATAAAAATTCATTTCATACGTTTTATAACTTATGCCCCTAAGTAT
TTTTTGACCATAGTGTTTCAATTCTACATTAATTTTACAGAGTAGAATGAAACGCCACCTACTCAGCCAA
GAGGCGAAAAGGTTAGCTCGCCAAGCAGAGAGGGCGCCAGTGCTCACTACTTTTTATAATTCTCAACTTC
TTTTTCCAGACTCAGTTCGTATATATAGACCTATTTTCAATTTAACGTCGCGCCGCCGACAATCCGAATG
GTTTTAGAGCTAGAAATAGCAAGTTAAAATAAGGCTAGTCCGTTATCAACTTGAAAAAGTGGCACCGAGT
CGGTGCTTTTTTGCCTACCTGGAGCCTGAGAGTTGTTCAATAAAATAAAAATGTTTCGTTTTTTTGCTTT
CGCCAGTATTTATTATGTACCTCTAGAGATCCACTAGTGTCGACGATGTAGGTCACGGTCTCGAAGCCGC
GGTGCGGGTGCCAGGGCGTGCCCTTGGGCTCCCCGGGCGCGTACTCCACCTCACCCATCTGGTCCATCAT
GATGAACGGGTCGAGGTGGCGGTAGTTGATCCCGGCGAACGCGCGGCGCACCGGGAAGCCCTCGCCCTCG
AAACCGCTGGGCGCGGTGGTCACGGTGAGCACGGGACGTGCGACGGCGTCGGCGGGTGCGGATACGCGGG
GCAGCGTCAGCGGGTTCTCGACGGTCACGGCGGGCATGTCGACACTAGTTCTAGCCAGCTTTTGTTCCCT
TTAGTGAGGGTTAATTTCGAGCTTGGCGTAATCATGGTCATAGCTGTTTCCTGTGTGAAATTGTTATCCG
CTCACAATTCCACACAACATACGAGCCGGAAGCATAAAGTGTAAAGCCTGGGGTGCCTAATGAGTGAGCT
AACTCACATTAATTGCGTTGCGCTCACTGCCCGCTTTCCAGTCGGGAAACCTGTCGTGCCAGCTGCATTA
ATGAATCGGCCAACGCGCGGGGAGAGGCGGTTTGCGTATTGGGCGCTCTTCCGCTTCCTCGCTCACTGAC
TCGCTGCGCTCGGTCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGCCAGGAACCGTAAAAA
GGCCGCGTTGCTGGCGTTTTTCCATAGGCTCCGCCCCCCTGACGAGCATCACAAAAATCGACGCTCAAGT
CAGAGGTGGCGAAACCCGACAGGACTATAAAGATACCAGGCGTTTCCCCCTGGAAGCTCCCTCGTGCGCT
CTCCTGTTCCGACCCTGCCGCTTACCGGATACCTGTCCGCCTTTCTCCCTTCGGGAAGCGTGGCGCTTTC
TCATAGCTCACGCTGTAGGTATCTCAGTTCGGTGTAGGTCGTTCGCTCCAAGCTGGGCTGTGTGCACGAA
CCCCCCGTTCAGCCCGACCGCTGCGCCTTATCCGGTAACTATCGTCTTGAGTCCAACCCGGTAAGACACG
ACTTATCGCCACTGGCAGCAGCCACTGGTAACAGGATTAGCAGAGCGAGGTATGTAGGCGGTGCTACAGA
GTTCTTGAAGTGGTGGCCTAACTACGGCTACACTAGAAGGACAGTATTTGGTATCTGCGCTCTGCTGAAG
CCAGTTACCTTCGGAAAAAGAGTTGGTAGCTCTTGATCCGGCAAACAAACCACCGCTGGTAGCGGTGGTT
TTTTTGTTTGCAAGCAGCAGATTACGCGCAGAAAAAAAGGATCTCAAGAAGATCCTTTGATCTTTTCTAC
GGGGTCTGACGCTCAGTGGAACGAAAACTCACGTTAAGGGATTTTGGTCATGAGATTATCAAAAAGGATC
TTCACCTAGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCATCCATAGT
TGCCTGACTCCCCGTCGTGTAGATAACTACGATACGGGAGGGCTTACCATCTGGCCCCAGTGCTGCAATG
ATACCGCGAGACCCACGCTCACCGGCTCCAGATTTATCAGCAATAAACCAGCCAGCCGGAAGGGCCGAGC
GCAGAAGTGGTCCTGCAACTTTATCCGCCTCCATCCAGTCTATTAATTGTTGCCGGGAAGCTAGAGTAAG
TAGTTCGCCAGTTAATAGTTTGCGCAACGTTGTTGCCATTGCTACAGGCATCGTGGTGTCACGCTCGTCG
TTTGGTATGGCTTCATTCAGCTCCGGTTCCCAACGATCAAGGCGAGTTACATGATCCCCCATGTTGTGCA
AAAAAGCGGTTAGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTGTGACTGGTGAG
TACTCAACCAAGTCATTCTGAGAATAGTGTATGCGGCGACCGAGTTGCTCTTGCCCGGCGTCAATACGGG
ATAATACCGCGCCACATAGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACCCAACTGATCTTCAGCA
TCTTTTACTTTCACCAGCGTTTCTGGGTGAGCAAAAACAGGAAGGCAAAATGCCGCAAAAAAGGGAATAA
GGGCGACACGGAAATGTTGAATACTCATACTCTTCCTTTTTCAATATTATTGAAGCATTTATCAGGGTTA
TTGTCTCATGAGCGGATACATATTTGAATGTATTTAGAAAAATAAACAAATAGGGGTTCCGCGCACATTT
CCCCGAAAAGTGCCAC
SEQ ID NO: 4-wild-type promoter sequence
ggccggcgggggeggttcgaagegggagcga
SEQ ID NO: 5-Ppyr* promoter sequence
ggccgg
SEQ ID NO: 6-wild-type promoter sequence
gttggccgcattcggattgtcggcggcggtt
SEQ ID NO: 7 0 Ppyr* promoter sequence
Cggcggcggtt
SEQ ID NO: 8-pMM7-8-1
GGCCCGGTACGTACCCAATTCGCCCTATAGTGAGTCGTATTACAATTCACTGGCCGTCGTTTTACAACGT
CGTGACTGGGAAAACCCTGGCGTTACCCAACTTAATCGCCTTGCAGCACATCCCCCTTTCGCCAGCTGGC
GTAATAGCGAAGAGGCCCGCACCGATCGCCCTTCCCAACAGTTGCGCAGCCTGAATGGCGAATGGAAATT
GTAAGCGTTAATATTTTGTTAAAATTCGCGTTAAATTTTTGTTAAATCAGCTCATTTTTTAACCAATAGG
CCGAAATCGGCAAAATCCCTTATAAATCAAAAGAATAGACCGAGATAGGGTTGAGTGTTGTTCCAGTTTG
GAACAAGAGTCCACTATTAAAGAACGTGGACTCCAACGTCAAAGGGCGAAAAACCGTCTATCAGGGCGAT
GGCCCACTACGTGAACCATCACCCTAATCAAGTTTTTTGGGGTCGAGGTGCCGTAAAGCACTAAATCGGA
ACCCTAAAGGGAGCCCCCGATTTAGAGCTTGACGGGGAAAGCCGGCGAACGTGGCGAGAAAGGAAGGGAA
GAAAGCGAAAGGAGCGGGCGCTAGGGCGCTGGCAAGTGTAGCGGTCACGCTGCGCGTAACCACCACACCC
GCCGCGCTTAATGCGCCGCTACAGGGCGCGTCAGGTGGCACTTTTCGGGGAAATGTGCGCGGAACCCCTA
TTTGTTTATTTTTCTAAATACATTCAAATATGTATCCGCTCATGAGACAATAACCCTGATAAATGCTTCA
ATAATATTGAAAAAGGAAGAGTATGAGTATTCAACATTTCCGTGTCGCCCTTATTCCCTTTTTTGCGGCA
TTTTGCCTTCCTGTTTTTGCTCACCCAGAAACGCTGGTGAAAGTAAAAGATGCTGAAGATCAGTTGGGTG
CACGAGTGGGTTACATCGAACTGGATCTCAACAGCGGTAAGATCCTTGAGAGTTTTCGCCCCGAAGAACG
TTTTCCAATGATGAGCACTTTTAAAGTTCTGCTATGTGGCGCGGTATTATCCCGTATTGACGCCGGGCAA
GAGCAACTCGGTCGCCGCATACACTATTCTCAGAATGACTTGGTTGAGTACTCACCAGTCACAGAAAAGC
ATCTTACGGATGGCATGACAGTAAGAGAATTATGCAGTGCTGCCATAACCATGAGTGATAACACTGCGGC
CAACTTACTTCTGACAACGATCGGAGGACCGAAGGAGCTAACCGCTTTTTTGCACAACATGGGGGATCAT
GTAACTCGCCTTGATCGTTGGGAACCGGAGCTGAATGAAGCCATACCAAACGACGAGCGTGACACCACGA
TGCCTGTAGCAATGGCAACAACGTTGCGCAAACTATTAACTGGCGAACTACTTACTCTAGCTTCCCGGCA
ACAATTAATAGACTGGATGGAGGCGGATAAAGTTGCAGGACCACTTCTGCGCTCGGCCCTTCCGGCTGGC
TGGTTTATTGCTGATAAATCTGGAGCCGGTGAGCGTGGGTCTCGCGGTATCATTGCAGCACTGGGGCCAG
ATGGTAAGCCCTCCCGTATCGTAGTTATCTACACGACGGGGAGTCAGGCAACTATGGATGAACGAAATAG
ACAGATCGCTGAGATAGGTGCCTCACTGATTAAGCATTGGTAACTGTCAGACCAAGTTTACTCATATATA
CTTTAGATTGATTTAAAACTTCATTTTTAATTTAAAAGGATCTAGGTGAAGATCCTTTTTGATAATCTCA
TGACCAAAATCCCTTAACGTGAGTTTTCGTTCCACTGAGCGTCAGACCCCGTAGAAAAGATCAAAGGATC
TTCTTGAGATCCTTTTTTTCTGCGCGTAATCTGCTGCTTGCAAACAAAAAAACCACCGCTACCAGCGGTG
GTTTGTTTGCCGGATCAAGAGCTACCAACTCTTTTTCCGAAGGTAACTGGCTTCAGCAGAGCGCAGATAC
CAAATACTGTCCTTCTAGTGTAGCCGTAGTTAGGCCACCACTTCAAGAACTCTGTAGCACCGCCTACATA
CCTCGCTCTGCTAATCCTGTTACCAGTGGCTGCTGCCAGTGGCGATAAGTCGTGTCTTACCGGGTTGGAC
TCAAGACGATAGTTACCGGATAAGGCGCAGCGGTCGGGCTGAACGGGGGGTTCGTGCACACAGCCCAGCT
TGGAGCGAACGACCTACACCGAACTGAGATACCTACAGCGTGAGCTATGAGAAAGCGCCACGCTTCCCGA
AGGGAGAAAGGCGGACAGGTATCCGGTAAGCGGCAGGGTCGGAACAGGAGAGCGCACGAGGGAGCTTCCA
GGGGGAAACGCCTGGTATCTTTATAGTCCTGTCGGGTTTCGCCACCTCTGACTTGAGCGTCGATTTTTGT
GATGCTCGTCAGGGGGGCGGAGCCTATGGAAAAACGCCAGCAACGCGGCCTTTTTACGGTTCCTGGCCTT
TTGCTGGCCTTTTGCTCACATGTTCTTTCCTGCGTTATCCCCTGATTCTGTGGATAACCGTATTACCGCC
TTTGAGTGAGCTGATACCGCTCGCCGCAGCCGAACGACCGAGCGCAGCGAGTCAGTGAGCGAGGAAGCGG
AAGAGCGCCCAATACGCAAACCGCCTCTCCCCGCGCGTTGGCCGATTCATTAATGCAGCTGGCACGACAG
GTTTCCCGACTGGAAAGCGGGCAGTGAGCGCAACGCAATTAATGTGAGTTAGCTCACTCATTAGGCACCC
CAGGCTTTACACTTTATGCTTCCGGCTCGTATGTTGTGTGGAATTGTGAGCGGATAACAATTTCACACAG
GAAACAGCTATGACCATGATTACGCCAAGCTCGAAATTAACCCTCACTAAAGGGAACAAAAGCTGGCTAG
AACTAGTGTCGACATGCCCGCCGTGACCGTCGAGAACCCGCTGACGCTGCCCCGCGTATCCGCACCCGCC
GACGCCGTCGCACGTCCCGTGCTCACCGTGACCACCGCGCCCAGCGGTTTCGAGGGCGAGGGCTTCCCGG
TGCGCCGCGCGTTCGCCGGGATCAACTACCGCCACCTCGACCCGTTCATCATGATGGACCAGATGGGTGA
GGTGGAGTACGCGCCCGGGGAGCCCAAGGGCACGCCCTGGCACCCGCACCGCGGCTTCGAGACCGTGACC
TACATCGTCGACACTAGTGGATCCAGCGGCCGCACCTGCAGGCCGGCCGTTAACACGCGTCCGCGGTCTA
GACTCGAGGATTCCAGATCTGGTACCGGGCCGCTGTATGGATATTTGCAGGGCCGCAGAATTCTCTCTAT
CACTGATAGGGAGGTCTCTATCACTGATAGGGAGTTCTCTATCACTGATAGGGATGTCTCTATCACTGAT
AGGGATTTCTCTATCACTGATAGGGAAGTCTCTATCACTGATAGGGACCTCTCTATCACTGATAGGGAAA
TCTCTATCACTGATAGGGATCTCTCTATCACTGATAGGGACTTCTCTATCACTGATAGGGACGTCTCTAT
CACTGATAGGGAACTCTCTATCACTGATAGGGACATCTCTATCACTGATAGGGACTTCTCTATCACTGAT
AGGGAAGTATCTTCTCTCTCTTCTCTTCTCTCTCTCTTTCTCGAATGTTCTCTCTCTTCTCTTCTCTCTC
TCTTTCTCGATGGCCGGGGCGCGCCAGGTTTCGACTTTCACTTTTCTCTATCACTGATAGGGAGTGGTAA
ACTCGACTTTCACTTTTCTCTATCACTGATAGGGAGTGGTAAACTCGACTTTCACTTTTCTCTATCACTG
ATAGGGAGTGGTAAACTCGACTTTCACTTTTCTCTATCACTGATAGGGAGATCCGAGCTCGTAAACTCGA
CTTTCACTTTTCTCTATCACTGATAGGGAGTGGTAAACTCGACTTTCACTTTTCTCTATCACTGATAGGG
AGTGGTAAACTCGACTTTCACTTTTCTCTATCACTGATAGGGAGTGGTAAACTCGAACGGATCCGTCGAG
GGAAAAGAGCGCCGGAGTATAAATAGAGGCGCTTCGTCTACGGAGCGACAATTCAATTCAAACAAGCAAA
GTGAACACGTCGCTAAGCGAAAGCTAAGCAAATAAACAAGCGCAGCTGAACAAGCTAAACAATCTGCAGC
CATGGTAATTTTCTTTACGTATATCAAGTGTTACGGCTCCATTTTTCTTTAGATATTTCCAGTATAGTTT
TTTATTACCAACATTTAAAAACAAATTTTAGAAAGCATACTGTTGGGATTTAAATGATTTTTTTATTAAA
AAGTGAGACAAAATTTTCAATACAGTTTTAATAATGGCAAAAGAAAATATACTGAAAACGTTGCATTTTC
CAAGAGGAACAAACTACAATCAACATACTATGTCTTGGTTTGAAGAAGAAGTTGTGACATATTGGGCAGT
TAAAACAAGACTATAACAGTGAGTATTATAAAAAAATTGTTAAAATAACATATTCCTATATATATTTATA
GCATTTTAAATAAATATTAAACATTTATTTATCATTAAGTTATAAGACATATATTCAAATATTGTTGTAA
CAGCTGTAAAAACAAGTTAGTTAATTGTTATTATTCAGGTTCTGGTTAAACTCCAGGTCATGAAATTGTG
TCTCTTCTATAAGATAAGTCCCTGGATCTTCTGGAGTAAGAGTGGTAGGATTGGCTTATCAGTTAAACAA
ATGTTCTGTATCATGTGGTTTGACCACATACTGGACAAATATTAGGGACGGCACGGTCAATACGAGACTA
GTATGCATCGAGACTATTGATCCACCCCAAGCGTAGTTGTTCCAGAGCTTCTTTTGTAGACCTCGGTAGA
TTCTGTTCTATAAAAACCAAGTATCATGCTTCTTACTGATGAGAATTAACGAGAACGTAAAGCGCCTACA
CATGTTTATAATGTATTCCCGCAAGCATATGTATACATTGTAATCTCTGTGTGACCATTATATTTATAAT
CTTTGATATAAACCTTATTCCAGGGTACTAAAGATATATTTTAATTTTATTTCTTTTGAATGTCTGTTGC
AAAATATGTAAATATATAAAAACTTTTATGAAAATATTATTTAAGTAAAGATATAAAATAGTTGAAAAAA
TCTTTGAATTGTAGAAAAATAGTCCGCTCCCCTACTTGATGCCCATATTCGCATTCGGTGTAGTCATCTT
GTTCCATGCTTTAAGCCGCTTCTCACAAGTATTCCAGTAAGAGGTCACCCGTGTTAATTAGGCATAGCTA
TCAGTTTATTTTAGCACTATTTGATCATCTAAATCCCTGCTCCTGCTAACTACCACCTTGTACTTAAAGT
ACATAAAATTTGGTCTTCAGCATCGTTCTTCGAGGGGCGGTCATAATATTTTTTATATTTTAAGGAGTGA
GATCGAACGTTTTTAAAGTGCTGTAATTTTGCTCGAATAGGTAGTACATCTCGTTTTAAAATCTAACACT
TGGAAACCTATTTTGTGCCCTTCAATTAATAATTATCATGAGCATAATATCTCTCTACTGGCCTTAGCTC
CGGGCTTTTTGGAGAAAAAAAGTCGGCACATAATGAAGTCTTATAAATGAAAACAGTCTTTCTTGTAAAC
GTTCCTTGATTTATATTTATAGAGCCTGTTGTAACAAATAATTAGCTTCTTAAAAAGAACTTGACTGATT
TTGGGTCCTAAATTTTTCTTCGACATTCTCTTGAGCATCAGCAACATAAAATTTTTTATTAGGTAGTTGC
AAAAAAACTGCCATGATTAGTCATCCATTATGCAACAAGAATACTTTTTCAAGAAAACTTGATTTTAATT
TCCGTACTCTGTTTTAGGCCTCTAATTTTTTGAGCAACATTCCTGTCAAAAACTTTTTGAGCCTTGTACG
TTTTTAAACCTGCATCAGCTTTAACTTTTCGTACCAAATAGTCCGACCAATGAGCTAACCGGGCTTTTCT
ACCTAATATGTTGGCAGCTCTTTTGAAAATATTTTGAGACATCATGTGAACCATTGCTTCCACATGAACC
AGTTTTCTCTGGGTACTGTTTGAAAAACATAGGAAACAGCTTGGCGGCAAACCTTTGAATGCTTGGGCAA
CTTTTGTGGGACAAAGTTTTGGTTTGTTGAAAATATTTAATAATTTATTAGGCACTTTTTTCTGGTCCCT
CATTTAAATCGGATTACCAATTTCATTTGTTTTTAATTAAACATTATAAATTATATGTTTTACATGTTTC
ACTAAACGTGTGTTACTAATTTTTCGATCTCACTCCTTTTATACCGTATTATATTAATTCTATACTGTAA
TTAAAGTTATTTTCAAATTGTTGCAATATTTATTTAGCAAAATGTTTTCATAACGTGAAATTGTATGTTA
TTTTTAGAAAAAATGTATATTAAGTTCTCAAATTCATATTGTTTATTCATTTAGATTCCCTTGAACAAAA
GGGGTTTGGTATTTTGTAAAAAATTACTATATTATTTAATAAGTAGTTAAGATTAATGAATTTCAGTGAA
TAATAAAAGCTCAACAATCAACATACTAACATTTTGAAGATCAGCAATATTAATCTATCAACACTAATTA
TAATTAACGACAATCAACATACCATAGAAAAAAGGATAATGGATAATGAATACAAAACTACAATCAACAT
TTTCCTCAGGGCAACACACATCTAGGTTTTGCAAGGATCAACAAATCAAGAGTGCATTAAAAATAACAAC
AATCAACATACCATAATTGAAGATGTTGCAAATATTGAAAATTTTTATTAAAAACATTTAAAATTTACTT
AAATTTTTCCTTTAAACGCAAATAAAAAGAAAACTTAAATTATTCTATTGCAAACAGAAAAAATCCCAAA
TTAAAATTTATTTAAAAATTATTTTTGTTATAAAACAAATCTAAAATCTATTTAATTTTAAAAATAATTA
AAAAAAAACATAAACGTGTTAAAACAATTTCACAGCTTAAAAATATCGATAAAAAATATATAATTTTTAA
TAATTTATTTTAATTAATCATCTTTATCAACATACAAAATGATAGATAGATTTTAAAAGGATCGAGGTTG
CATGTATGATAAATTTATTATTCTTTTCTATGTTTAGGTCAGCCGTTTGGATAAATCCAAAGTTATTAAT
TCCGCTTTGGAATTGTTGAATGAAGTTGGTATTGAAGGTTTGACAACACGTAAATTGGCTCAAAAATTGG
GTGTTGAACAACCAACATTGTATTGGCATGTTAAAAATAAACGTGCTTTGTTGGATGCTTTGGCTATTGA
AATGTTGGACCGTCATCATACACATTTTTGCCCATTGGAAGGCGAATCCTGGCAAGATTTCTTGCGTAAT
AATGCCAAATCCTTCCGTTGTGCTTTGTTGTCCCATCGTGATGGTGCCAAGGTTCATTTGGGCACACGTC
CAACAGAAAAACAATATGAAACATTGGAAAATCAATTGGCTTTCTTGTGTCAACAAGGCTTCAGCTTGGA
AAATGCTTTGTATGCTTTGAGCGCCGTTGGTCATTTTACATTGGGCTGTGTGTTGGAAGATCAAGAACAT
CAAGTCGCTAAAGAAGAACGTGAAACACCAACAACAGATTCGATGCCCCCATTGTTGCGTCAAGCAATTG
AATTGTTCGATCATCAAGGAGCCGAACCAGCATTCTTGTTCGGCTTGGAATTGATTATTTGTGGATTGGA
AAAACAATTGAAATGTGAATCGGGCTCGGGCCCCGCGTACAGCCGCGCGCGTACGAAAAACAATTACGGG
TCTACCATCGAGGGCCTGCTCGATCTCCCGGACGACGACGCCCCCGAAGAGGCGGGGCTGGCGGCTCCGC
GCCTGTCCTTTCTCCCCGCGGGACACACGCGCAGACTGTCGACGGCCCCCCCGACCGATGTCAGCCTGGG
GGACGAGCTCCACTTAGACGGCGAGGACGTGGCGATGGCGCATGCCGACGCGCTAGACGATTTCGATCTG
GACATGTTGGGGGACGGGGATTCCCCGGGTCCGGGATTTACCCCCCACGACTCCGCCCCCTACGGCGCTC
TGGATATGGCCGACTTCGAGTTTGAGCAGATGTTTACCGATGCCCTTGGAATTGACGAGTACGGTGGGTA
GTAAGCTTGGATCTTTGTGAAGGAACCTTACTTCTGTGGTGTGACATAATTGGACAAACTACCTACAGAG
ATTTAAAGCTCTAAGGTAAATATAAAATTTTTAAGTGTATAATGTGTTAAACTACTGATTCTAATTGTTT
GTGTATTTTAGATTCCAACCTATGGAACTGATGAATGGGAGCAGTGGTGGAATGCCTTTAATGAGGAAAA
CCTGTTTTGCTCAGAAGAAATGCCATCTAGTGATGATGAGGCTACTGCTGACTCTCAACATTCTACTCCT
CCAAAAAAGAAGAGAAAGGTAGAAGACCCCAAGGACTTTCCTTCAGAATTGCTAAGTTTTTTGAGTCATG
CTGTGTTTAGTAATAGAACTCTTGCTTGCTTTGCTATTTACACCACAAAGGAAAAAGCTGCACTGCTATA
CAAGAAAATTATGGAAAAATATTCTGTAACCTTTATAAGTAGGCATAACAGTTATAATCATAACATACTG
TTTTTTCTTACTCCACACAGGCATAGAGTGTCTGCTATTAATAACTATGCTCAAAAATTGTGTACCTTTA
GCTTTTTAATTTGTAAAGGGGTTAATAAGGAATATTTGATGTATAGTGCCTTGACTAGAGATCATAATCA
GCCATACCACATTTGTAGAGGTTTTACTTGCTTTAAAAAACCTCCCACACCTCCCCCTGAACCTGAAACA
TAAAATGAATGCAATTGTTGTTGTTAACTTGTTTATTGCAGCTTATAATGGTTACAAATAAAGCAATAGC
ATCACAAATTTCACAAATAAAGCATTTTTTTCACTGCATTCTAGTTGTGGTTTGTCCAAACTCATCAATG
TATCTTATCATGTCTCGAGCATGCGCAAATTTAAAGCGCTGATATCGATCGCGCGCAGATCTGTCATGAT
GATCATTGCAATTCTGCAGTCGACGGTACCCGATCTTGTCGCCGGAACGCAGCGACAGAGATTCCAATGT
GTCCGTATCTTTCAGGCTTTTGCCCTTCAGTTCCAGACGAAGCGACTGGCGATTCGCGTGTGGGGTCTGC
TTCAGGGTCTTGTGAATTAGGGCGCGCAGATCGCCGATGGGCGTGGCGCCGGAGGGCACCTTCACCTTGC
CGTACGGCTTGCTGTTCTTCGCGTTCAAAATCTCCAGCTCCATTTTGCTTTCGGTGCGCTTGCAATCAGT
ACTGTCCAAAATCGAAAATCGCCGAACCGTAGTGTGACCGTGCGGGGCTCTGCGAAAATAAACTTTTTTA
GGTATATGGCCACACACGGGGAAAGCACAGTGGATTATATGTTTTAATATTATAATATGCAGGTTTTCAT
TACTTATCCAGATGTAAGCCCACTTAAAGCGATTTAACAATTATTTGCCGAAAGAGTAAAAACAAATTTC
ACTTAAAAATGGATTAAGAAAAGCTTGTGTAAGATTATGCGCAGCGTTGCCAGATAGCTCCATTTAAAAC
ACTTCAAAAACAATAAGTTTTGAAAATATATACATAAATAGCAGTCGTTGCCGCAACGCTCAACACATCA
CACTTTTAAAACACCCTTTACCTACACAGAATTACTTTTTAAATTTCCAGTCAAGCTGCGAGTTTCAAAA
TTATAGCCGGTAGAGAAGACAGTGCTATTTCAAAAGCAAACTAAATAAACACCAATCCTAACAAGCCTTG
GACTTTTGTAAGTTTAGATCAAAGGTGGCATTGCATTCAATGTCATGGTAAGAAGTAGGTCGTCTAGGTA
GAAATCCTCATTCAGCCGGTCAAGTCAGTACGAGAAAGGTCTCAATTTGAAATTGTCTTAAAAATATTTT
ATTGTTTTGTACTGTGGTGAGTTTAAACGAAAAACACAAAAAAAAAGTGATACACAGAAATCATAAAAAA
TTTTAATACAAGGTATTCGTACGTATCAAAAACATTTCGGCACAATTTTTTTTCTCTGTACTAAAGTGTT
ACGAACACTACGGTATTTTTTAGTGATTTTCAACGGACACCGAAGGTATATAAACAGCGTTCGCGAACGG
TCGCCTTCAAAACCAATTGACATTTGCAGCAGCAAGTACAAGCAGAAAGTAAAGCGCAATCAGCGAAAAA
TTTATACTTAATTGTTGGTGATTAAAGTACAATTAAAAGAACATTCTCGAAAGTCACAAGAAACGTAAGT
TTTTAACTCGCTGTTACCAATTAGTAATAAGAGCAACAAGACGTTGAGTAATTTCAAGAAAAACTGCATT
TCAAGGTCTTTGTTCGGCCATTTTTTTTTTATTCAACGCTCTACGTAATTACAAAATAAGAAATTGGCAG
CCACGCATCTTGTTTTCCCAATCAATTGGCATCAAAACGCAAACAAATCTATAAATAAAACTTGCGTGTT
GATTTTCGCCAAGATTTATTGGCAAATTGTGAAATTCGCAGTGACGCATTTGAAAATTCGAGAAATCACG
AACGCACTCGAGCATTTGTGTGCATGTTATTAGTTAGTTAGTTCTTTGCTTAATTGAAGTATTTTACCAA
CGAAATCCACTTATTTTTAGCTGAAATAGAGTAGGTTGCTTGAAACGAAAGCCACGTCTGGAAAATTTCT
TATTGCTTAGTAGTTGTGACGTCACCATATACACACAAAATAATGTGTATGCATGCGTTTCAGCTGTGTA
TATATACATGCACACACTCGCATTATGAAAACGATGACGAGCAACGGAACAGGTTTCTCAACTACCTTTG
TTCCTGTTTCTTCGCTTTCCTTTGTTCCAATATTCGTAGAGGGTTAATAGGGGTTTCTCAACAAAGTTGG
CGTCGATAAATAAGTTTCCCATTTTTATTCCCCAGCCAGGAAGTTAGTTTCAATAGTTTTGTAATTTCAA
CGAAACTCATTTGATTTCGTACTAATTTTCCACATCTCTATTTTGACCCGCAGAATAATCCAAAATGCAG
ATCGGGGATCCCACCCCACCCAAGAAGAAGCGCAAGGTGGAGGACGATCCCGTCGTTTTACAACGTCGTG
ACTGGGAAAACCCTGGCGTTACCCAACTTAATCGCCTTGCAGCACATCCCCCTTTCGCCAGCTGGCGTAA
TAGCGAAGAGGCCCGCACCGATCGCCCTTCCCAACAGTTGCGGTCGACTCTAGAGGATCCCCGGGATCCA
CCGGTCGCCACCATGGTGAGCAAGGGCGAGGAGCTGTTCACCGGGGTGGTGCCCATCCTGGTCGAGCTGG
ACGGCGACGTAAACGGCCACAAGTTCAGCGTGTCCGGCGAGGGCGAGGGCGATGCCACCTACGGCAAGCT
GACCCTGAAGTTCATCTGCACCACCGGCAAGCTGCCCGTGCCCTGGCCCACCCTCGTGACCACCCTGACC
TACGGCGTGCAGTGCTTCAGCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAGACCCGCGCCGAGGT
GAAGTTCGAGGGCGACACCCTGGTGAACCGCATCGAGCTGAAGGGCATCGACTTCAAGGAGGACGGCAAC
ATCCTGGGGCACAAGCTGGAGTACAACTACAACAGCCACAACGTCTATATCATGGCCGACAAGCAGAAGA
ACGGCATCAAGGTGAACTTCAAGATCCGCCACAACATCGAGGACGGCAGCGTGCAGCTCGCCGACCACTA
CCAGCAGAACACCCCCATCGGCGACGGCCCCGTGCTGCTGCCCGACAACCACTACCTGAGCACCCAGTCC
GCCCTGAGCAAAGACCCCAACGAGAAGCGCGATCACATGGTCCTGCTGGAGTTCGTGACCGCCGCCGGGA
TCACTCTCGGCATGGACGAGCTGTACAAGTAAAGCGGCCGCGACTCTAGATCATAATCAGCCATACCACA
TTTGTAGAGGTTTTACTTGCTTTAAAAAACCTCCCACACCTCCCCCTGAACCTGAAACATAAAATGAATG
CAATTGTTGTTGTTAACTTGTTTATTGCAGCTTATAATGGTTACAAATAAAGCAATAGCATCACAAATTT
CACAAATAAAGCATTTTTTTCACTGCATTCTAGTTGTGGTTTGTCCAAACTCATCAATGTATCTTAAAGC
TTATCGATACGCGTACGGCACTAGAGCGGCCGCCACCGCGGTGGAGCTCCAGCTTTTGTTCCCTTTAGTG
AGGGTTAATTAGATCGGCCGGCCTTGGCGCGCCTAGATCTTAATACGACTCACTATAGGGCGAATTGGGT
ACCG

1. A system for controlling population of a biological species comprising a first sex and a second sex, the system comprising:
genetically-modified individuals comprising:
a repressible genetic mutation that results in:
death of a juvenile individual of the first sex when the juvenile individual of the first sex comprises the repressible lethal mutation and is reared in the absence of a repressor; or
sterility of an individual of the first sex when the individual of the first sex comprises the repressible lethal mutation and is reared in the absence of a repressor; and
an underdominant genetic mutation so that:
offspring of a mating between a member of the system and a wild-type member of the biological species are nonviable; and
offspring of a mating between two members of the system results in viable offspring only of the second sex.

2. The system of claim 1, wherein the first sex is female and the repressible genetic mutation comprises a female lethal mutation.

3. The system of claim 1, wherein the first sex is male and the repressible genetic mutation comprises a male lethal mutation.

4. The system of claim 1, wherein the underdominant mutation comprises a synthetic incompatibility mutation.

5. The system of claim 1, wherein the biological species comprises an invasive species.

6. The system of claim 1, wherein the biological species comprises a pest species.

7. The system of claim 1, wherein the biological species comprises an insect.

8. The system of claim 1, wherein the biological species comprises a fish.

9. A method for controlling population of a wild biological species, the method comprising:
releasing into a wild population of the wild biological species a system for controlling the population of the wild biological species, the system comprising:
a population of genetically-modified individuals of the biological species, the population comprising a first sex and a second sex, the genetically-modified individuals comprising:
a repressible genetic mutation that results in:
death of a juvenile individual of the first sex when the juvenile individual of the first sex comprises the repressible lethal mutation and is reared in the absence of a repressor; or
sterility of an individual of the first sex when the individual of the first sex comprises the repressible lethal mutation and is reared in the absence of a repressor; and
an underdominant genetic mutation so that:
offspring of a mating between a member of the system and a wild-type member of the biological species are nonviable; and
offspring of a mating between two members of the system results in viable offspring only of the second sex; and
allowing members of the system subpopulations mate with wild members of the biological species in the absence of the repressor.

10. The method of claim 9, wherein the first sex is female and the repressible genetic mutation comprises a female lethal mutation.

11. The method of claim 9, wherein the first sex is male and the repressible genetic mutation comprises a male lethal mutation.

12. The method of claim 9, wherein the underdominant mutation comprises an engineered genetic incompatibility mutation.

13. The method of claim 9, wherein the combination of the repressible lethal mutation and the underdominant mutation reduces the population the wild biological species synergistically compared to using either the repressible mutation alone or the underdominant mutation alone.


SEQUENCE LISTING (8 sequences)

SEQ ID NO: 1 - DNA, 6869 bp, Artificial
  plasmid
  misc_feature (5)..(5): n is a, c, g, or t
  misc_feature (45)..(45): n is a, c, g, or t
  misc_feature (78)..(78): n is a, c, g, or t
  misc_feature (80)..(80): n is a, c, g, or t
ccacncacgtttcgtagttgctctttcgctgtctcccacccgctntccgcaacacattcaccttttgttcgacgaccntnggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcggccgcaatcttacaaaatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacacta

SEQ ID NO: 2 - DNA, 9370 bp, Artificial
  plasmid
  misc_feature (5)..(5): n is a, c, g, or t
  misc_feature (45)..(45): n is a, c, g, or t
  misc_feature (78)..(78): n is a, c, g, or t
  misc_feature (80)..(80): n is a, c, g, or t
ccacncacgtttcgtagttgctctttcgctgtctcccacccgctntccgcaacacattcaccttttgttcgacgaccntnggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgtcaaatttggttgtgattacgagacggagaccgagacggcgacgacagttagccattcgccacgcgccaacgcaaatgaaacgctctatacatatttttgtatattttctgtttttttttgccgctgacaattatgatcaagtattagctggcgatagctgaaacgtctgtgtaatttcaatggaatggaatgggaagtgggcggcctattgatacactgctcgagtgattttaacttttatctgatcattcaaacgcataaattagtcttgagaacttcaattcatttgatactccagttaacatgctatttacatgctcatttaaatggtagtagtgatttataagcccacttccagatggaacttacctataccaacgtgttacttatcgttcttaagccaacttaatagcattctaaaatatatatgtatcttttggcggacttatcttcttgttgttctcgcattccaaaatctctatgtacatgcaaacttttattgtcataactcgggactttgcagactttgaggcctatttaatagagctataatcttacaacaaaaaaaaactaaaagagctttttaagcaataaaaatattctgaaaaattacaaattaacaaaaaattacccaatgaagcctgcaaatttgaaatctttaagatcctagatatgccaagatgcaccctaaagtccttaactcatctccttggctcgtttctaatcccccctctcgagggatcgagacgatcgcatcgggtcggtctttaagtttggatgatccataaactgttggtttctccgtcctcagcgtctagacttcattagccgtgtaatgttgcggaatttatgtggcaggcacattaaaataacaccgatacacactctcatggacgcgaacgtgtgtacaagtatagagatatcgggcctaggcgaaaaatgaaattaaaaaaaaaaaaaaaactggcaccgggaggggcttatttttcggtggtcggggatgcgggggactttgaccataaaatacatgctcccaaaaagctcgcacactgcaagagatgcggggcacttctgagtcccatattcatatgcacaaatgtgcattgctggcattatcagtagaatgcaatttcgggaaattttccatcgcatcacgagacaatgaacgtaagagagaaatggagcctcaaagagggagggagagagagagcttgagtgaacgagcgagcgacaatcgcgagataacggctgccttatcagcaatgccgaccgccccatcaccccacccaaaacgcccaaccaccacccaccgccgccgttccctttcctccatcgtcgagaatttcgagttcagagcagcgcgaccgaaatgaaaagaaacaatttaaattccaaatgtataaaataggtaaactatggcttttatttattaatattgacgggggcacaaaggcggtcacctcaatagtgaataacatgttttttataatgaatacttttcaaattgttattaatatgcaatgacgtcttaaatgtttcactgcagctgaactttattcttttcattaaaacagtcacccgttattaaaaataaatagattaagttttatattattaaatttgtaagtattgaaacaattccttttttattttatatgaattatcatttagttggggttaatatcccttaaagagagaaatttgtatgctttaagatttaaaatatctattgcatttatagctatagctataacttctcttatttcacgcagaaaatactcaaataaaacatatcgatttggcataccccactaattttttggccccaagtgtgtgagagtgtgtgaggcgaagcgcgccacaaacataaaaaagcggtgaagtgagcggttgtggaacgtgagtggatgctaagagcaagctctcacatacgcggacataggtcgcacacacacacgcacagaccgcctttttgcgccgccgaaacgaacacttttacgaaggcgacggcgaatcagtttcagttgtcagttcgcatccaactagaaagcagttaacgagtagtctgtgttttttcgcttgcggttaaaagccacgaggtcgttcatcgttcatcgttttccttttcaacttcaagcaaagcaaatataaaccaatgcaaaaaacgcagtgatcttttgaggcccaaatcgtttggggccgaacaccgttgattctaaaacgcaaatgtagaaacaaatcaagaaagtggaaaataaatatgtttcgctttcaaaacatgtgaatgtgccgaactcaaaactgaaacgtagaaggaacgcgttcgttttttacatacgacaatcgtataaaataagagaaaagctccaaaacgtattaaatagcgatgcttggatgatcttcgtagcagtcacgttgtacatacaaatacatacatatgtacctactatatggcacataaaatacgttacgcacactagtggcgaataaaaagcgaattggagcaatcttacaaaatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacacta

SEQ ID NO: 3 - DNA, 17866 bp, Artificial
  plasmid
ctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtacgtaccgggcccctagtatgtatgtaagttaataaaacccatttttgcggaaagtagataaaaaaaacattttttttttttactgcactggatatcattgaacttatctgatcagttttaaatttacttcgatccaagggtatttgatgtaccaggttctttcgattacctctcactcaaaatgacattccactcaaagtcagcgctgtttgcctccttctctgtccacagaaatatcgccgtctctttcgccgctgcgtccgctatctctttcgccaccgtttgtagcgttacgtagcgtcaatgtccgccttcagttgcattttgtcagcggtttcgtgacgaagctccaagcggtttacgccatcaattaaacacaaagtgctgtgccaaaactcctctcgcttcttatttttgtttgttttttgagtgattggggtggtgattggttttgggtgggtaagcaggggaaagtgtgaaaaatcccggcaatgggccaagaggatcaggagctattaattcgcggaggcagcaaacacccatctgccgagcatctgaacaatgtgagtagtacatgtgcatacatcttaagttcacttgatctataggaactgcgattgcaacatcaaattgtctgcggcgtgagaactgcgacccacaaaaatcccaaaccgcaattgcacaaacaaatagtgacacgaaacagattattctggtagctgttctcgctatataagacaatttttgagatcatatcatgatcaagacatctaaaggcattcattttcgactatattcttttttacaaaaaatataacaaccagatattttaagctgatcctagatgcacaaaaaataaataaaagtataaacctacttcgtaggatacttcggggtactttttgttcggggttagatgagcataacgcttgtagttgatatttgagatcccctatcattgcagggtgacagcggagcggcttcgcagagctgcattaaccagggcttcgggcaggccaaaaactacggcacgctccggccacccagtccgccggaggactccggttcagggagcggccaactagccgagaacctcacctatgcctggcacaatatggacatctttggggcggtcaatcagccgggctccggatggcggcagctggtcaaccggacacgcggactattctgcaacgagcgacacataccggcgcccaggaaacatttgctcaagaacggtgagtttctattcgcagtcggctgatctgtgtgaaatcttaataaagggtccaattaccaatttgaaactcagtttgcggcgtggcctatccgggcgaacttttggccgtgatgggcagttccggtgccggaaagacgaccctgctgaatgcccttgcctttcgatcgccgcagggcatccaagtatcgccatccgggatgcgactgctcaatggccaacctgtggacgccaaggagatgcaggccaggtgcgcctatgtccagcaggatgacctctttatcggctccctaacggccagggaacacctgattttccaggccatggtgcggatgccacgacatctgacctatcggcagcgagtggcccgcgtggatcaggtgatccaggagctttcgctcagcaaatgtcagcacacgatcatcggtgtgcccggcagggtgaaaggtctgtccggcggagaaaggaagcgtctggcattcgcctccgaggcactaaccgatccgccgcttctgatctgcgatgagcccacctccggactggactcatttaccgcccacagcgtcgtccaggtgctgaagaagctgtcgcagaagggcaagaccgtcatcctgaccattcatcagccgtcttccgagctgtttgagctctttgacaagatccttctgatggccgagggcagggtagctttcttgggcactcccagcgaagccgtcgacttcttttcctagtgagttcgatgtgtttattaagggtatctagcattacattacatctcaactcctatccagcgtgggtgcccagtgtcctaccaactacaatccggcggacttttacgtacaggtgttggccgttgtgcccggacgggagatcgagtcccgtgatcggatcgccaagatatgcgacaattttgctattagcaaagtagcccgggatatggagcagttgttggccaccaaaaatttggagaagccactggagcagccggagaatgggtacacctacaaggccacctggttcatgcagttccgggcggtcctgtggcgatcctggctgtcggtgctcaaggaaccactcctcgtaaaagtgcgacttattcagacaacggtgagtggttccagtggaaacaaatgatataacgcttacaattcttggaaacaaattcgctagattttagttagaattgcctgattccacacccttcttagtttttttcaatgagatgtatagtttatagttttgcagaaaataaataaatttcatttaactcgcgaacatgttgaagatatgaatattaatgagatgcgagtaacattttaatttgcagatggttgccatcttgattggcctcatctttttgggccaacaactcacgcaagtgggcgtgatgaatatcaacggagccatcttcctcttcctgaccaacatgacctttcaaaacgtctttgccacgataaatgtaagtcttgtttagaatacatttgcatattaataatttactaactttctaatgaatcgattcgatttaggtgttcacctcagagctgccagtttttatgagggaggcccgaagtcgactttatcgctgtgacacatactttctgggcaaaacgattgccgaattaccgctttttctcacagtgccactggtcttcacggcgattgcctatccgatgatcggactgcgggccggagtgctgcacttcttcaactgcctggcgctggtcactctggtggccaatgtgtcaacgtccttcggatatctaatatcctgcgccagctcctcgacctcgatggcgctgtctgtgggtccgccggttatcataccattcctgctctttggcggcttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggccattctcatcgtgagcttccgggtgctcgcatatctggctctaagacttcgggcccgacgcaaggagtagccgacatatatccgaaataactgcttgttttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggttcgctcaaatggttccgagtggttcatttcgtctcaatagaaattagtaataaatatttgtatgtacaatttatttgctccaatatatttgtatatatttccctcacagctatatttattctaatttaatattatgactttttaaggtaattttttgtgacctgttcggagtgattagcgttacaatttgaactgaaagtgacatccagtgtttgttccttgtgtagatgcatctcaaaaaaatggtgggcataatagtgttgtttatatatatcaaaaataacaactataataataagaatacatttaatttagaaaatgcttggatttcactggaactaggctagcataacttcgtataatgtatgctatacgaagttatgctagcggatccgggaattgggaattcacgtaagtactgtctgcagcgtaagcttcgtacgtagcgtcaaatttggttgtgattacgagacggagaccgagacggcgacgacagttagccattcgccacgcgccaacgcaaatgaaacgctctatacatatttttgtatattttctgtttttttttgccgctgacaattatgatcaagtattagctggcgatagctgaaacgtctgtgtaatttcaatggaatggaatgggaagtgggcggcctattgatacactgctcgagtgattttaacttttatctgatcattcaaacgcataaattagtcttgagaacttcaattcatttgatactccagttaacatgctatttacatgctcatttaaatggtagtagtgatttataagcccacttccagatggaacttacctataccaacgtgttacttatcgttcttaagccaacttaatagcattctaaaatatatatgtatcttttggcggacttatcttcttgttgttctcgcattccaaaatctctatgtacatgcaaacttttattgtcataactcgggactttgcagactttgaggcctatttaatagagctataatcttacaacaaaaaaaaactaaaagagctttttaagcaataaaaatattctgaaaaattacaaattaacaaaaaattacccaatgaagcctgcaaatttgaaatctttaagatcctagatatgccaagatgcaccctaaagtccttaactcatctccttggctcgtttctaatcccccctctcgagggatcgagacgatcgcatcgggtcggtctttaagtttggatgatccataaactgttggtttctccgtcctcagcgtctagacttcattagccgtgtaatgttgcggaatttatgtggcaggcacattaaaataacaccgatacacactctcatggacgcgaacgtgtgtacaagtatagagatatcgggcctaggcgaaaaatgaaattaaaaaaaaaaaaaaaactggcaccgggaggggcttatttttcggtggtcggggatgcgggggactttgaccataaaatacatgctcccaaaaagctcgcacactgcaagagatgcggggcacttctgagtcccatattcatatgcacaaatgtgcattgctggcattatcagtagaatgcaatttcgggaaattttccatcgcatcacgagacaatgaacgtaagagagaaatggagcctcaaagagggagggagagagagagcttgagtgaacgagcgagcgacaatcgcgagataacggctgccttatcagcaatgccgaccgccccatcaccccacccaaaacgcccaaccaccacccaccgccgccgttccctttcctccatcgtcgagaatttcgagttcagagcagcgcgaccgaaatgaaaagaaacaatttaaattccaaatgtataaaataggtaaactatggcttttatttattaatattgacgggggcacaaaggcggtcacctcaatagtgaataacatgttttttataatgaatacttttcaaattgttattaatatgcaatgacgtcttaaatgtttcactgcagctgaactttattcttttcattaaaacagtcacccgttattaaaaataaatagattaagttttatattattaaatttgtaagtattgaaacaattccttttttattttatatgaattatcatttagttggggttaatatcccttaaagagagaaatttgtatgctttaagatttaaaatatctattgcatttatagctatagctataacttctcttatttcacgcagaaaatactcaaataaaacatatcgatttggcataccccactaattttttggccccaagtgtgtgagagtgtgtgaggcgaagcgcgccacaaacataaaaaagcggtgaagtgagcggttgtggaacgtgagtggatgctaagagcaagctctcacatacgcggacataggtcgcacacacacacgcacagaccgcctttttgcgccgccgaaacgaacacttttacgaaggcgacggcgaatcagtttcagttgtcagttcgcatccaactagaaagcagttaacgagtagtctgtgttttttcgcttgcggttaaaagccacgaggtcgttcatcgttcatcgttttccttttcaacttcaagcaaagcaaatataaaccaatgcaaaaaacgcagtgatcttttgaggcccaaatcgtttggggccgaacaccgttgattctaaaacgcaaatgtagaaacaaatcaagaaagtggaaaataaatatgtttcgctttcaaaacatgtgaatgtgccgaactcaaaactgaaacgtagaaggaacgcgttcgttttttacatacgacaatcgtataaaataagagaaaagctccaaaacgtattaaatagcgatgcttggatgatcttcgtagcagtcacgttgtacatacaaatacatacatatgtacctactatatggcacataaaatacgttacgcacactagtggcgaataaaaagcgaattggagcaatcttacaaaatggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttgaccgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgcccaaactcatcaatgtatcttagcggctcgagggtacaagccgaattgatccactagaaggcctaattcggtacactagttggccacgtaataagtgtgcgttgaatttattcgcaaaaacattgcatattttcggcaaagtaaaattttgttgcataccttatcaaaaaataagtgctgcatactttttagagaaaccaaataattttttattgcatacccgtttttaataaaatacattgcataccctcttttaataaaaaatattgcatactttgacgaaacaaattttcgttgcatacccaataaaagattattatattgcatacccgtttttaataaaatacattgcataccctcttttaataaagaatattgcatacgttgacgaaacaaattttcgttgcatacccaataaaagattattatattgcataccttttcttgccataccatttagccgatcaattgagatctgaattcattttcaacgtcctcgatagtatagtggttagtatccccgcctgtcacgcgggagaccggggttcaattccccgtcggggagaatctgtgattctttttttttttcttttactttgttatataaacaatttttgttttaattgaatctaatttgccattgcttttaggaatctcaggcatccagcaagcgtttgtccgccgaatcgcccatcagtgaagaagatcctgtggcggctacgaaaatctccccggccatgtcggcctccacctccagcgaaaaacccatcagcgagctggccacctctgtgctgacccaccgctttccagactccacctcctcacccggcgaacatggccttggacgaatgcagttgtcgatccgctacagcgcccagcgtcaaaaactagacgtgaccatacacaaaatccagaagataccacttcgcgatcccagcaatatccccgatccgtatgttaagctgtatctgttgcctggacgcaccaaggagtcgaaacgcaagacgagcgtgatcaaggacaactgcaaccccgtctacgatgcatcctttgagtacctgatttccattgccgaactcaggcagacggaactggaggtgacggtgtgcacccaaaagggattcctatccggcggtagtcccatcattggcatggtaggtacccgaaagcaaccccttagttacagacacagcgcgtacgtccttcgcatccttatgattcccaagtacatattctgcaagagtacagtatatataggaaagatatccgggtgaacttcgcggcgggggcggttcgaagcgttttagagctagaaatagcaagttaaaataaggctagtccgttatcaacttgaaaaagtggcaccgagtcggtgcttttttgctcacctgtgattgctcctactcaaatacaaaaacatcaaattttctgtcaataaagcatatttatttatatttattttacaggaaagaattccttttaaagtgtattttaacctataatgaaaaacgattaaaaaaaatacataaaataattcgaaaatttttgaatagcccaggttgataaaaattcatttcatacgttttataacttatgcccctaagtattttttgaccatagtgtttcaattctacattaattttacagagtagaatgaaacgccacctactcagccaagaggcgaaaaggttagctcgccaagcagagagggcgccagtgctcactactttttataattctcaacttctttttccagactcagttcgtatatatagacctattttcaatttaacgtcgcgccgccgacaatccgaatggttttagagctagaaatagcaagttaaaataaggctagtccgttatcaacttgaaaaagtggcaccgagtcggtgcttttttgcctacctggagcctgagagttgttcaataaaataaaaatgtttcgtttttttgctttcgccagtatttattatgtacctctagagatccactagtgtcgacgatgtaggtcacggtctcgaagccgcggtgcgggtgccagggcgtgcccttgggctccccgggcgcgtactccacctcacccatctggtccatcatgatgaacgggtcgaggtggcggtagttgatcccggcgaacgcgcggcgcaccgggaagccctcgccctcgaaaccgctgggcgcggtggtcacggtgagcacgggacgtgcgacggcgtcggcgggtgcggatacgcggggcagcgtcagcgggttctcgacggtcacggcgggcatgtcgacactagttctagccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 4 - DNA, 31 bp, Drosophila Melanogaster
ggccggcgggggcggttcgaagcgggagcga

SEQ ID NO: 5 - DNA, 6 bp, Drosophila Melanogaster
ggccgg

SEQ ID NO: 6 - DNA, 31 bp, Drosophila Melanogaster
gttggccgcattcggattgtcggcggcggtt

SEQ ID NO: 7 - DNA, 11 bp, Drosophila Melanogaster
cggcggcggtt

SEQ ID NO: 8 - DNA, 12394 bp, Artificial
  plasmid
ggcccggtacgtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggctagaactagtgtcgacatgcccgccgtgaccgtcgagaacccgctgacgctgccccgcgtatccgcacccgccgacgccgtcgcacgtcccgtgctcaccgtgaccaccgcgcccagcggtttcgagggcgagggcttcccggtgcgccgcgcgttcgccgggatcaactaccgccacctcgacccgttcatcatgatggaccagatgggtgaggtggagtacgcgcccggggagcccaagggcacgccctggcacccgcaccgcggcttcgagaccgtgacctacatcgtcgacactagtggatccagcggccgcacctgcaggccggccgttaacacgcgtccgcggtctagactcgaggattccagatctggtaccgggccgctgtatggatatttgcagggccgcagaattctctctatcactgatagggaggtctctatcactgatagggagttctctatcactgatagggatgtctctatcactgatagggatttctctatcactgatagggaagtctctatcactgatagggacctctctatcactgatagggaaatctctatcactgatagggatctctctatcactgatagggacttctctatcactgatagggacgtctctatcactgatagggaactctctatcactgatagggacatctctatcactgatagggacttctctatcactgatagggaagtatgttctctctcttctcttctctctctctttctcgaatgttctctctcttctcttctctctctctttctcgatggccggggcgcgccaggtttcgactttcacttttctctatcactgatagggagtggtaaactcgactttcacttttctctatcactgatagggagtggtaaactcgactttcacttttctctatcactgatagggagtggtaaactcgactttcacttttctctatcactgatagggagatccgagctcgtaaactcgactttcacttttctctatcactgatagggagtggtaaactcgactttcacttttctctatcactgatagggagtggtaaactcgactttcacttttctctatcactgatagggagtggtaaactcgaacggatccgtcgagggaaaagagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagccatggtaattttctttacgtatatcaagtgttacggctccatttttctttagatatttccagtatagttttttattaccaacatttaaaaacaaattttagaaagcatactgttgggatttaaatgatttttttattaaaaagtgagacaaaattttcaatacagttttaataatggcaaaagaaaatatactgaaaacgttgcattttccaagaggaacaaactacaatcaacatactatgtcttggtttgaagaagaagttgtgacatattgggcagttaaaacaagactataacagtgagtattataaaaaaattgttaaaataacatattcctatatatatttatagcattttaaataaatattaaacatttatttatcattaagttataagacatatattcaaatattgttgtaacagctgtaaaaacaagttagttaattgttattattcaggttctggttaaactccaggtcatgaaattgtgtctcttctataagataagtccctggatcttctggagtaagagtggtaggattggcttatcagttaaacaaatgttctgtatcatgtggtttgaccacatactggacaaatattagggacggcacggtcaatacgagactagtatgcatcgagactattgatccaccccaagcgtagttgttccagagcttcttttgtagacctcggtagattctgttctataaaaaccaagtatcatgcttcttactgatgagaattaacgagaacgtaaagcgcctacacatgtttataatgtattcccgcaagcatatgtatacattgtaatctctgtgtgaccattatatttataatctttgatataaaccttattccagggtactaaagatatattttaattttatttcttttgaatgtctgttgcaaaatatgtaaatatataaaaacttttatgaaaatattatttaagtaaagatataaaatagttgaaaaaatctttgaattgtagaaaaatagtccgctcccctacttgatgcccatattcgcattcggtgtagtcatcttgttccatgctttaagccgcttctcacaagtattccagtaagaggtcacccgtgttaattaggcatagctatcagtttattttagcactatttgatcatctaaatccctgctcctgctaactaccaccttgtacttaaagtacataaaatttggtcttcagcatcgttcttcgaggggcggtcataatattttttatattttaaggagtgagatcgaacgtttttaaagtgctgtaattttgctcgaataggtagtacatctcgttttaaaatctaacacttggaaacctattttgtgcccttcaattaataattatcatgagcataatatctctctactggccttagctccgggctttttggagaaaaaaagtcggcacataatgaagtcttataaatgaaaacagtctttcttgtaaacgttccttgatttatatttatagagcctgttgtaacaaataattagcttcttaaaaagaacttgactgattttgggtcctaaatttttcttcgacattctcttgagcatcagcaacataaaattttttattaggtagttgcaaaaaaactgccatgattagtcatccattatgcaacaagaatactttttcaagaaaacttgattttaatttccgtactctgttttaggcctctaattttttgagcaacattcctgtcaaaaactttttgagccttgtacgtttttaaacctgcatcagctttaacttttcgtaccaaatagtccgaccaatgagctaaccgggcttttctacctaatatgttggcagctcttttgaaaatattttgagacatcatgtgaaccattgcttccacatgaaccagttttctctgggtactgtttgaaaaacataggaaacagcttggcggcaaacctttgaatgcttgggcaacttttgtgggacaaagttttggtttgttgaaaatatttaataatttattaggcacttttttctggtccctcatttaaatcggattaccaatttcatttgtttttaattaaacattataaattatatgttttacatgtttcactaaacgtgtgttactaatttttcgatctcactccttttataccgtattatattaattctatactgtaattaaagttattttcaaattgttgcaatatttatttagcaaaatgttttcataacgtgaaattgtatgttatttttagaaaaaatgtatattaagttctcaaattcatattgtttattcatttagattcccttgaacaaaaggggtttggtattttgtaaaaaattactatattatttaataagtagttaagattaatgaatttcagtgaataataaaagctcaacaatcaacatactaacattttgaagatcagcaatattaatctatcaacactaattataattaacgacaatcaacataccatagaaaaaaggataatggataatgaatacaaaactacaatcaacattttcctcagggcaacacacatctaggttttgcaaggatcaacaaatcaagagtgcattaaaaataacaacaatcaacataccataattgaagatgttgcaaatattgaaaatttttattaaaaacatttaaaatttacttaaatttttcctttaaacgcaaataaaaagaaaacttaaattattctattgcaaacagaaaaaatcccaaattaaaatttatttaaaaattatttttgttataaaacaaatctaaaatctatttaattttaaaaataattaaaaaaaaacataaacgtgttaaaacaatttcacagcttaaaaatatcgataaaaaatatataatttttaataatttattttaattaatcatctttatcaacatacaaaatgatagatagattttaaaaggatcgaggttgcatgtatgataaatttattattcttttctatgtttaggtcagccgtttggataaatccaaagttattaattccgctttggaattgttgaatgaagttggtattgaaggtttgacaacacgtaaattggctcaaaaattgggtgttgaacaaccaacattgtattggcatgttaaaaataaacgtgctttgttggatgctttggctattgaaatgttggaccgtcatcatacacatttttgcccattggaaggcgaatcctggcaagatttcttgcgtaataatgccaaatccttccgttgtgctttgttgtcccatcgtgatggtgccaaggttcatttgggcacacgtccaacagaaaaacaatatgaaacattggaaaatcaattggctttcttgtgtcaacaaggcttcagcttggaaaatgctttgtatgctttgagcgccgttggtcattttacattgggctgtgtgttggaagatcaagaacatcaagtcgctaaagaagaacgtgaaacaccaacaacagattcgatgcccccattgttgcgtcaagcaattgaattgttcgatcatcaaggagccgaaccagcattcttgttcggcttggaattgattatttgtggattggaaaaacaattgaaatgtgaatcgggctcgggccccgcgtacagccgcgcgcgtacgaaaaacaattacgggtctaccatcgagggcctgctcgatctcccggacgacgacgcccccgaagaggcggggctggcggctccgcgcctgtcctttctccccgcgggacacacgcgcagactgtcgacggcccccccgaccgatgtcagcctgggggacgagctccacttagacggcgaggacgtggcgatggcgcatgccgacgcgctagacgatttcgatctggacatgttgggggacggggattccccgggtccgggatttaccccccacgactccgccccctacggcgctctggatatggccgacttcgagtttgagcagatgtttaccgatgcccttggaattgacgagtacggtgggtagtaagcttggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctcgagcatgcgcaaatttaaagcgctgatatcgatcgcgcgcagatctgtcatgatgatcattgcaattctgcagtcgacggtacccgatcttgtcgccggaacgcagcgacagagattccaatgtgtccgtatctttcaggcttttgcccttcagttccagacgaagcgactggcgattcgcgtgtggggtctgcttcagggtcttgtgaattagggcgcgcagatcgccgatgggcgtggcgccggagggcaccttcaccttgccgtacggcttgctgttcttcgcgttcaaaatctccagctccattttgctttcggtgcgcttgcaatcagtactgtccaaaatcgaaaatcgccgaaccgtagtgtgaccgtgcggggctctgcgaaaataaacttttttaggtatatggccacacacggggaaagcacagtggattatatgttttaatattataatatgcaggttttcattacttatccagatgtaagcccacttaaagcgatttaacaattatttgccgaaagagtaaaaacaaatttcacttaaaaatggattaagaaaagcttgtgtaagattatgcgcagcgttgccagatagctccatttaaaacacttcaaaaacaataagttttgaaaatatatacataaatagcagtcgttgccgcaacgctcaacacatcacacttttaaaacaccctttacctacacagaattactttttaaatttccagtcaagctgcgagtttcaaaattatagccggtagagaagacagtgctatttcaaaagcaaactaaataaacaccaatcctaacaagccttggacttttgtaagtttagatcaaaggtggcattgcattcaatgtcatggtaagaagtaggtcgtctaggtagaaatcctcattcagccggtcaagtcagtacgagaaaggtctcaatttgaaattgtcttaaaaatattttattgttttgtactgtggtgagtttaaacgaaaaacacaaaaaaaaagtgatacacagaaatcataaaaaattttaatacaaggtattcgtacgtatcaaaaacatttcggcacaattttttttctctgtactaaagtgttacgaacactacggtattttttagtgattttcaacggacaccgaaggtatataaacagcgttcgcgaacggtcgccttcaaaaccaattgacatttgcagcagcaagtacaagcagaaagtaaagcgcaatcagcgaaaaatttatacttaattgttggtgattaaagtacaattaaaagaacattctcgaaagtcacaagaaacgtaagtttttaactcgctgttaccaattagtaataagagcaacaagacgttgagtaatttcaagaaaaactgcatttcaaggtctttgttcggccattttttttttattcaacgctctacgtaattacaaaataagaaattggcagccacgcatcttgttttcccaatcaattggcatcaaaacgcaaacaaatctataaataaaacttgcgtgttgattttcgccaagatttattggcaaattgtgaaattcgcagtgacgcatttgaaaattcgagaaatcacgaacgcactcgagcatttgtgtgcatgttattagttagttagttctttgcttaattgaagtattttaccaacgaaatccacttatttttagctgaaatagagtaggttgcttgaaacgaaagccacgtctggaaaatttcttattgcttagtagttgtgacgtcaccatatacacacaaaataatgtgtatgcatgcgtttcagctgtgtatatatacatgcacacactcgcattatgaaaacgatgacgagcaacggaacaggtttctcaactacctttgttcctgtttcttcgctttcctttgttccaatattcgtagagggttaataggggtttctcaacaaagttggcgtcgataaataagtttcccatttttattccccagccaggaagttagtttcaatagttttgtaatttcaacgaaactcatttgatttcgtactaattttccacatctctattttgacccgcagaataatccaaaatgcagatcggggatcccaccccacccaagaagaagcgcaaggtggaggacgatcccgtcgttttacaacgtcgtgactgggaaaaccctggcgttacccaacttaatcgccttgcagcacatccccctttcgccagctggcgtaatagcgaagaggcccgcaccgatcgcccttcccaacagttgcggtcgactctagaggatccccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttatcgatacgcgtacggcactagagcggccgccaccgcggtggagctccagcttttgttccctttagtgagggttaattagatcggccggccttggcgcgcctagatcttaatacgactcactatagggcgaattgggtaccg